(19) United States
(12) Patent Application Publication
(10) Pub. No.: US 2010/0100160 A1
(43) Pub. Date: Apr. 22, 2010

(54) Methods and devices for self adjusting phototherapeutic intervention

(75) Inventors: Carl Frederick Edman, San Diego, CA (US); Naresh Chandra Bhavaraju, San Diego, CA (US); Darrel Dean Drinan, San Diego, CA (US)

Correspondence Address: Carl Edman;c/o PhiloMetron Inc., Suite 100, 10451 Roselle St., San Diego, CA, 92121, US

(73) Assignee: PhiloMetron, Inc., San Diego, CA (US)

(21) Appl. No.: 12/589,014

(22) Filed: Oct. 16, 2009

Related U.S. Application Data
(60) Provisional application No. 61/196,491, filed on Oct. 16, 2008.

(51) Int. Cl. A61N 5/06

(52) U.S. Cl. 607/88

(57) ABSTRACT

The present invention relates to devices for the sensing of one or more body regions within a larger body zone, and the application of a therapy, preferably photonic therapy, based upon one or more sensor measurements derived from at least one body region. Such applied photonic therapies may be accomplished in an effectively simultaneous fashion while being adjusted independently to each body region. In addition, successive applications of photonic therapy(s) to one or more body regions may be further adjusted based upon one or more successive sensor measurements of the corresponding body regions.

### CROSS REFERENCE TO RELATED PATENTS

This application claims priority under 35 U.S.C. Section 119(e) to provisional application No. 61/196,491, filed on Oct. 16, 2008.

### FEDERALLY SPONSORED RESEARCH OR DEVELOPMENT

Not Applicable

### BACKGROUND OF THE INVENTION

Photonic based therapeutic activities employ the direct application of light, typically of one or more wavelengths in either the ultraviolet, visible or near infrared regions, to remedy an undesired physiological condition. Photonic therapies may also employ the use of one or more photosensitive agents as part of therapy. To date, these activities generally require the observational skills of the clinician, typically in conjunction with one or more diagnostic imaging tools, in order to assess the location and efficacy of the applied photonic therapy.

For instance, low level laser therapy apparatus (U.S. Pat. No. 6,312,451) teaches the use of a handheld apparatus for delivering photonic energies of defined wavelengths for the purpose of beneficial clinical effects. However, said invention while disclosing preferred methods of operation and use, does not disclose methods and means enabling automatic identification and/or quantification of body conditions that would benefit from the use of the apparatus.

Quantification of body region status and/or therapy status would be highly desirable so that tissues or body regions not requiring phototherapy may avoid receiving such therapy whereas regions that may benefit may receive one or more phototherapies tailored to said regions. Consider a phototherapy bandage of rectangular shape such as that described by U.S. Pat. No. 7,304,201 placed over an incisional wound of narrow, oblong dimensions. Segments of healthy tissue will necessarily be covered by the bandage. These covered healthy segments may not benefit and possibly may be injured by misapplication of phototherapy to these regions. Conversely, the tissue injury regions or portions thereof that might benefit from the application of such therapy may receive inadequate therapy if such therapy is provided based upon the totality of the tissue covered.

Altshuler and Tuchin (U.S. Pat. No. 7,329,273) teach the use of sensors for the purpose of providing diagnostic information regarding oral tissue to receive photonic radiation treatment as well as to provide the user with information regarding completion of the treatment session, etc. However, they do not teach differential adjustment of phototreatment to simultaneously treated oral structures based upon sensor measurements nor the use of a plurality of sensors combined with a plurality of photodelivery in order to accomplish said differential treatment.

Mager, et al. (U.S. Pat. No. 5,944,748) teach the use of combining sensors to delineate lesion areas from surrounding tissue followed by selective application of photodynamic therapy to the lesion area. However, while teaching the use of signal modulation and frequency selective filters within detection circuits to minimize unwanted signals arising from stray light, they fail to teach methods to prevent signal illumination crosstalk occurring between individual excitation photodiodes and respective photocells for the purpose of sensing during such modulated processes or individually addressable sensor elements. Nor does this invention teach the use of differentially modulating phototherapy within the identified lesion area. Thus, this invention falls short of the need for phototherapeutic systems adaptable to a variety of sensors and/or applications.

In related art, there exist numerous descriptions of the use of one or more photosensitizer agents that when irradiated by light of appropriate wavelengths and intensities generate compounds producing a phototherapeutic response or activity, e.g. cancer cell destruction. In order to increase the effectiveness of such photosensitizers, methods for the selective targeting of photoagents, e.g. U.S. Pat. No. 6,894,161 describes the crosslinking of photoagents to target molecules or tissues, have been described. As a somewhat alternative approach, U.S. Pat. No. 5,435,307 describes the use of fluorescent signals enabling the quantification of photosensitizer agents in a desired location. However, these approaches, while enabling the application of the agents, do not provide objective automated processes for determining region(s) of the body for treatment and thereby avoiding unnecessary and potentially harmful treatments on healthy body regions. These arts also do not provide quantitative assessment of the effectiveness of the treatment thereby enabling adjustment to subsequent treatments or doses.

Therefore, there exists a need to provide identification and quantitative assessment of body regions that would benefit from the use of phototherapies coupled with the delivery of phototherapies and thereby improving phototherapy treatments.

### SUMMARY OF THE INVENTION

The invention described herein presents the method and devices for enabling the measurement and identification of one or more body regions for the subsequent application of therapy and enabling targeted therapy delivery to one or more identified body regions. In the context of this invention, the body region may be on the body surface, including the skin and/or body cavities such as the ear, mouth, vagina, uterus or anus, or the body region may be located within the body including tissues, bones, muscle groups, digestive tract, etc.

In a preferred form of the present invention, the structure for enabling identification of one or more body regions for subsequent therapeutic treatment is comprised of a plurality of sensing technologies (sensors) enabling automated inspection of a still larger portion of the body termed a body zone such that this zone encompass one or more body regions. In preferred embodiments, the present invention may be substantially flexible and planar in structure, e.g. a patch-like structure, having a first surface with a plurality of sensors incorporated within its structure and intended to be positioned towards or against the body zone to be inspected. Such sensors may encompass a substantial portion of the first surface area and may, in certain instances, effectively comprise the entire first surface area.

In other embodiments, the structure for identification is comprised of one or more sensors that are utilized by passage over, focused at, through or around a body zone in order to identify one or more body regions that would benefit from therapeutic treatment. In still other forms of the invention, the system of the invention is incorporated within other medical devices or structures, e.g. clothing, catheter, stents, dressings, chairs, beds, etc. such that the method of the invention may be accomplished in a body zone adjacent to at least a portion of the device.

Within the present invention, data from one or more said sensors may be automatically processed by at least one comparator and regions of the body identified for subsequent therapeutic activity. Such processing may include comparative processing of scanned zones for the purpose of distinguishing between regions that might not benefit from therapy as compared to those regions that might do so. Upon identification, locations of such regions that may benefit from therapy may be displayed and/or otherwise indicated to a device user. In addition, in certain embodiments of the invention, these locations may be automatically transferred to one or more therapy delivering structures. In certain embodiments of the invention, therapeutic activity may then be commenced either automatically or, in a preferred embodiment, upon clinician command to one or more so identified body regions.

In the context of the present invention, a preferred therapeutic activity involves the transmission of at least one therapeutic energy into an identified body region. Such energies may include but are not limited to, photonic, acoustic, mechanical, electromagnetic electrical energies, or combinations of one or more energies. In a preferred form of the present invention, such energies are photonic in nature, and may encompass one or more wavelengths within the ultraviolet, visible or infrared spectrum. Therapies may also include the delivery of one or more therapeutic agents, drugs, plasmas, and/or other forms of therapy, e.g. debridement, to one or more identified body regions, as part of the method and system of this invention.

In a related embodiment of the present invention, the identification process is repeated following delivery of the therapy enabling adjustment of subsequent therapy or treatments. Recommended adjustment of therapy may occur also as part of the initial body region identification and location transfer to the therapeutic delivery structure.

### BRIEF DESCRIPTION OF THE DRAWINGS

FIG. 1—General representation of the present invention.

FIG. 2—Illustration of a preferred embodiment of the present invention.

FIG. 3—Representation of one embodiment of the present invention for the calibration of photonic emissions (cross sectional view).

FIG. 4—Illustration of a planar photonic sensor array according to the method of the present invention.

FIG. 5—Cross sectional view illustrating non-desired photonic passage between elements of a sensor array (Panel A) and illustrating various forms of the invention for the removal or minimization of such passage (Panels B and C).

FIG. 6—Cross sectional view illustrating signal complexity arising from simultaneous activation of multiple photonic sensors (Panel A) and illustrating one form of the present invention for solving such complexity (Panel B).

FIG. 7—Cross sectional view illustrating various embodiments of the present invention for the differential inspection of tissue depths through use of spaced photonic light sources (Panel A) or sensors (Panel B).

FIG. 8—One embodiment a first surface of the present invention having both sensor elements and photonic therapy delivery sources.

FIG. 9—Outline of one embodiment of comparator activities according to the method of the present invention.

FIG. 10—Illustration of one embodiment of the present invention adapted for use in dental therapy.

FIG. 11—Illustration of one embodiment of the present invention intended to be moved over a body zone of interest.

FIG. 12—Illustration of one embodiment of the present invention intended to be positioned about a vascular structure and/or device.

FIG. 13—Cross sectional view illustrating an embodiment of the present invention also enabling a second function as a wound dressing.

### DETAILED DESCRIPTION OF THE INVENTION

The present invention generally relates to the use of one or more sensing technologies enabling determination of at least one body region that would benefit from one or more therapeutic treatments within a larger body zone, the automatic identification of the location of said body region by the device or data derived by measurements of device of the present invention and the subsequent employment of said determined location in the delivery of one or more therapies to the identified body region. Within the scope of the present invention, sensing and/or therapeutic deliveries may be accomplished in a periodic or continuous fashion or ad hoc upon instruction or command.

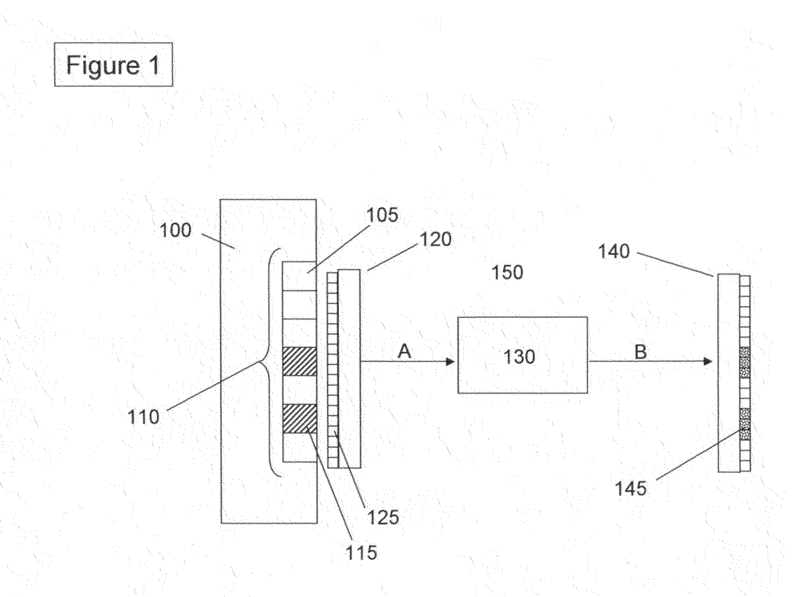

A general illustration the invention is shown FIG. 1. As shown, device 150 has at least one measurement structure 120 having multiple sensor elements, exemplified by single sensor 125 for measuring body zone 110 on body 100. Said measurements enable the determination of at least one body region 115 that would benefit from at least one therapy. Also shown is at least one comparator 130 for conducting the analysis of measurement data received (indicated by arrow A) from the measurement structure 120 and at least one therapy delivery structure 140 for the targeted delivery of therapy, indicated by activated region 145 of one or more therapies to the intended body region(s). Said targeted therapy is resultant from information conveyed by comparator 130 to therapy delivery structure 140, as shown by arrow B.

In a preferred embodiment, the measurement structure 120 has a plurality of sensors enabling the inspection of a body zone 110 without the need to reposition the structure about the body zone in order to accomplish the intended sensing. In such embodiments the measurement structure may be substantially planar in form, e.g. constructed as a patch or flexible sheet, and having a first surface from which sensors energies may be transmitted and/or received thereby enabling sensor measurements to be made. In other embodiments, the measurement structure may be incorporated into medical devices such as catheters, implanted structures, etc. In yet still other embodiments, the structure for enabling inspection of a body zone has at least one sensor but is located within, moved or positioned about a body zone to enable the determination of one or more regions that would benefit from the applied therapy.

In order to utilize the information received from one or more sensors, the device of the present invention has at least one comparator 130 to enable the determination of one or more body regions that would benefit from therapy. In a preferred embodiment, the comparator has at least one display means, e.g. visual (such as visual display or flashing lights), audible (such as instructions from an automated artificial voice or sounds) or mechanical (such as vibrations), indicating the presence of a body region that would benefit from therapeutic delivery. In alternative embodiments, the comparator stores data received from the sensors and then assembles the data in a representative display, e.g. map, of the body region indicating those regions that would benefit from application of one or more therapies.

Finally, the device of the present invention has at least one therapeutic delivery component or structure 140. Such therapies may consist in whole or in part of energies, e.g. photonic energies, ultrasonic energies, or electrical energies, delivered to one or more identified body regions. Such embodiments may also include photonic therapies utilizing one or more light responsive agents, e.g. photosensitizers, as part of the therapeutic activity. In alternate embodiments, such therapies may consist of the selective delivery of one or more agents or drugs to a targeted body region. In still other embodiments, a combination of energies, drugs or other therapies may be administered to one or more identified body regions.

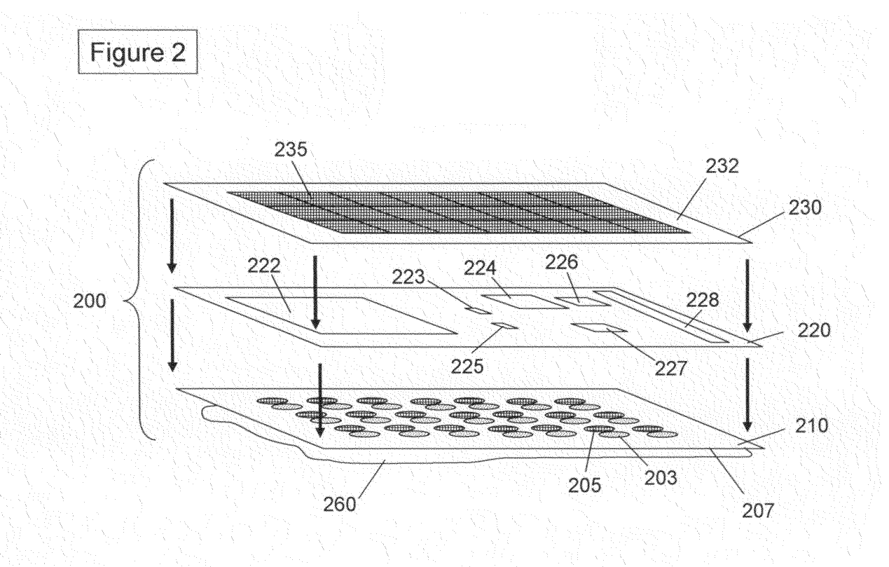

The preferred embodiment of a device of the present invention is a substantially planar and flexible, thereby facilitating substantive conformal contact of the device with a body zone. The preferred embodiment incorporates sensor structure, comparator and therapy delivery functionalities within a single overall structure. An illustration of a preferred embodiment of the present invention is shown in FIG. 2, wherein three major layers or segments of the device 200 are separated for purpose of illustration but intended in practice to be formed into a single, substantially planar conformal device (indicated by bold downward pointing arrows). In most applications, the overall planar dimensions, i.e. dimensions describing the lengths of contact of the first surface to a body zone, are generally between 5 cm and 30 cm on any one edge, in order to provide useful coverage of a body zone, however, other dimensions or shapes, e.g. circular, are readily conceivable within the scope of the present invention.

Within the structure of device 200, a plurality of sensing light sources and photodetectors combined into single entities, e.g. paired assemblies having one source and one photodetector, indicated by 203 are shown arranged on a lower layer 210 having first surface 207. A second set of light sources 205 useful for delivery of phototherapy are also shown arrayed on the first surface. Said sensing light sources, photodetectors and light sources may encompass a substantial portion of the first surface in order to accomplish the desired sensing and therapy delivery activities. In addition, said repetitive arrangements of light sources and detectors enable the area defined by surface 207 to be readily divided into regions thereby facilitating measurement and therapeutic treatment of a body zone.

Middle layer 220 illustrates controlling circuitry including switch 225, amplifier 223, processor 224, memory 227, wireless transceiver 226, antenna 228, and power source such as that supplied by printed battery 222. Electrical interconnecting traces and vias are not shown but are assumed to be present, as needed, in order to provide necessary circuitry connections. Said controlling circuitry enables sensor function, comparator function, communication function, display function and phototherapy delivery function and the skills to design and construct such circuitry is well known to those skilled in the art of electronic circuitry construction. Upper layer 230 contains multiple display elements 235 arranged on second surface 232.

Said display elements may be utilized to convey information regarding the measured zone, e.g. provide representations of data associated with regions as measured by photonic elements 203 present on first surface 207. In this representation, said display elements are substantially in the form of a matrix array, however other forms of display, e.g. single light sources, numeric value displays or even wireless communication of status may utilized within the scope of the present invention. Also shown is adhesive 260 that is located on at least a portion of first surface 207 to aid in affixing first surface to a body zone to be measured. Alternative methods to affix or position the device against a body zone are conceivable, e.g. use of straps, and the invention is not limited to the employment of adhesives.

To provide electrical connections between the functions located on the various layers of device 200, which enables device, vias containing electrically connective materials or epoxies may be employed, however other forms of electrical connections, e.g. wires, may be utilized within the scope of the present invention to this purpose. In yet other forms of the present invention, photonic elements are utilized to provide communication and/or power between various portions or elements of the present device, e.g. the use of light sources and photodetectors to transfer the electrical signal and or communicate information between the separated structures such as sensors and comparators not in direct physical contact or where electrical contacts may be disrupted by body fluids, etc.

To construct the circuitry elements of device 200, e.g. one or circuit elements, such as switch 225, antenna 228 and printed battery 222, electrical traces connecting these elements, or the light sources and detectors 203, advantageous use may be made of one or more one or more methods of electronic manufacture employing printed circuitry or printed electronics. Said printed electronic technology fabricates electrically functional elements directly on the substrate through the printing appropriate materials onto a substrate as compared to traditional forms of electronic circuitry construction wherein said components are separable discrete units placed at desired location on a circuit board then electrically connected to one or more electrical traces to enable functionality. Printed circuitry may enable increased flexibility of the overall structure of device 200 as well as provide lower cost of fabrication as compared to other methods of circuit construction.

Other circuit elements may be discrete circuit components which are arranged at desired locations and then electrically connected through use of conductive epoxies or other forms of electrical bonding. Such components may include processor 224 or other elements requiring functionalities, e.g. speed of operation, not necessarily attainable using available printed electronic components.

In one form of this embodiment, removal of device 200 from sealed packaging and exposure to air, light, tissue, or tissue fluids may trigger the function one or more circuit elements to initiate device activity, i.e. to turn the device “on”. In other forms, a supply of energy transmitted to the antenna may serve to initiate device activity. Multiple methods to activate the device are conceivable and the scope of the invention is not limited to these methods presented herein.

Once activated, device 200 may periodically take sensor measurement data utilizing preprogrammed instructions and store measured values. These measured data and/or analysis characterizing the measured body regions within the inspected zone may then be displayed either periodically, upon determination that measurements from one or more regions exceeding preset parameters, or upon command, e.g. signals transmitted to the receiving antenna to initiate display activities. In addition, said data and analysis in certain embodiments may be wirelessly transmitted to a local device that may display, further analyze, combine/analyze with other stored data or inputted data or store for future use and review. In response to said displayed or transmitted data and analysis, one or more therapeutic actions may be enabled to target one or more regions. Such enablement may be effectively automatic, i.e. performed by device 200 without external triggering or commands utilizing integrated therapy algorithms and instructions. Alternatively, therapy initiation may be initiated by external command, e.g. clinician activation, utilizing forms of feedback to device 200 possibly including wireless instruction and/or touch display commands.

Additional elements may be employed in the overall structure of device 200 to aid or expand the functionality of the device in the intended application. For instance, in applications such as wound healing monitoring, it may be desirable to include elements such as gauze or other absorbent materials within the structure of device 200. In such embodiments, a variety of wicking channels may be incorporated within the first surface 207 that would convey liquids or wound exudate from the wound to a layer of absorbent materials interposed between either lower layer 210 and middle layer 220 or between middle layer 220 and upper layer 230. Electrical connections between respective layers of device 200 may be made using traces or other forms of connection and are readily conceivable by those skilled in the art of electrical circuit construction. In such forms, device 200 may provide two functions, one function as a bandage for absorption of wound fluids and protection against further contamination of the wound surface and the second function as a monitor of the wound healing. In other forms, materials, e.g. gauze or other absorbents may be interposed between two or more optical sources or detectors on first surface 207. Such interpositions would be selected to enable sensing or therapeutic functions as well as wound exudate removal functions. These interposed materials may absorb wound exudate directly if no other absorbent materials are present. Alternatively, these materials may convey wound exudate to one or more additional layers of absorbent contained within or otherwise in substantive contact with device 200. Such devices may include use of vacuums or other mechanical means to remove fluid from the wound.

In related embodiments of the invention, one or more sensing or therapeutic agents, e.g. fluorescent visualization dyes, photosensitizers, antibiotics, etc., are substantially integrated or located in one or more elements within the structure of a device of the invention. In such embodiments, the agents may be selectively released, e.g. through the use of pumps, electroosmotic forces, electroactive polymers serving as pumps or valves, from one or more reservoirs as needed or upon command to one or more body regions. In yet other embodiments, one or more sensing or therapeutic agents is integrated into one or more structures, polymers or other material such that upon signal, e.g. an energy (applied light) resulting in photolysis, applied electrical signal resulting in electrosensitive release or enzymatic release, one or more sections of said structure or polymer is perturbed such that the sensing or therapeutic agent is released. In still other embodiments, the sensing and/or therapeutic agents may be released at a set rate to one or more body regions which is then titrated based upon sensor input and or comparator analysis.

In FIG. 2, three elements of the present invention are contained within a single structure. In other embodiments, the therapeutic delivery component is a physically separable element from the sensor and/or comparator elements. In yet other embodiments, the sensor element may also be separated into two or more separable elements, e.g. photonic sources may be located in structures physically separated from structures containing photodetectors. Such embodiments may be useful in those applications employing one or more implanted structures that upon illumination, results in the measurement of body regions within a body zone and then enables localized therapeutic responses to said analysis. An example of such application may be the use of the device in an implant intended to provide localized therapy, e.g. drug release, to body areas possibly experience cancer regrowth. By a measured change in a diagnostic marker, e.g. an increase in local vascularity, recurrence of the cancer may be detected and responded to by selective release of chemotherapeutic agents from the implanted portion of the device to that body region. Photonic illumination enabling said measurements and possibly serving as energy source for device activity may be supplied from one or more illumination sources located outside of the body surface.

In related embodiments, photonic illumination may serve to drive sensor, comparator and therapeutic delivery functions. In such embodiments, the photonic illumination may be a directed illumination or one arising from ambient or other light sources. Effective circuit components, including light responsive elements, e.g. photocells, as well as means to store received energy, e.g. rechargeable batteries and/or capacitors, may be incorporated into such devices. Yet other forms of the invention may utilize other energy sources, e.g. radiofrequency waves and/or electromagnetic radiations, and thereby enable replenishment of energy needed for device functionality.

In various embodiments of the present invention, the location information derived from the comparator may be automatically provided to the therapy delivery element thereby enabling improved targeting of the therapy.

In variations of the present invention, one or more structures may be incorporated to enable repeated use of the devices of the present invention on multiple body zones and/or individuals. One such structure may be a disposable biocompatible interface such as a sheet or film. In such embodiments, the biocompatible interface may have one or more elements of the present invention incorporated, e.g. light sources or photodetectors. In other embodiments, the biocompatible interface material may be constructed in such a fashion and of materials as to enable the functioning of sensing and/or therapy delivery e.g. light filters. In other embodiments, the device of the invention may be disposable. In general terms, use of a disposable device and/or structure of the device may aid in the prevention of transmission of infectious agents or materials from one body location to another or from one individual to another.

In other embodiments, the therapeutic device of the invention may also include one or more means of storing and selectively delivering one or more therapeutic agents to identified body regions. Such therapeutic agents may comprise one or more active agents or materials stored in a reservoir-like or integrated into the device and selectively dispensed to specific regions upon activation of one or more dispensing means, e.g. micropumps made from electroactive polymers.

In still other embodiments, the sensing structures and/or the therapeutic delivery structures may also include structures having other functionalities, e.g. absorbent materials for use as a wound dressing, incorporation into prosthetics or implanted medical devices, etc. Additional forms of the device are conceivable and the method and devices of the present invention are not restricted to the forms or embodiments presented here.

In yet other embodiments, the sensing and/or therapy delivery structures may be substantially incorporated as elements within a medical device or structure, e.g. a catheter line, enabling improved function of the medical device or structure. For example, the present invention may be included as a sheath or covering of a catheter line, utilizing a sole illumination source placed outside the body, transmitting at least a portion of light down said catheter line. Multiple photodetection elements interspersed about the line thereby enable detection/location of disruptive events within the line, e.g. occlusion, or on the outside of the line, e.g. infection and/or biofilm formation. Upon detection of such events, appropriate therapeutic intervention may be initiated, thereby improving overall medical device functionality.

Other embodiments of the device configured as a handheld device may have utility in use for ad-hoc or periodic monitoring of body zones by clinicians. In such embodiments, additional features may be present, e.g. notification or reminders to perform measurement activities, as well as one or more methods to include patient identification and/or inspected body zone with each measurement activity. Such data may be entered in by a keyboard, a scanner functionality reading a wrist band worn by the patient, etc. The scope of the invention is not limited by the method for including patient/body zone identifications and the synchronization of these identifications with one or more measurements.

In still other embodiments, the device may be incorporated into structures such as cell phones, beds, chairs, or bed pads, wherein such structures have a primary functionality unrelated to device use. In short, the device of the present invention may be configured in a variety of fashions and the scope of the present invention is not limited to the configurations presented here.

A more detailed description of each of the three principal elements of the present invention, i.e. sensors, comparator and therapy delivery, is presented below.

Sensors

In order to accomplish the method of the present invention, at least one sensor able to detect one or more signals from of a body region that would benefit by one or more applied therapies is utilized. Such regions may be the result of a disease condition, e.g. infection of a wound or the presence of cancer, the presence of altered healing, e.g. reaction to introduced materials or devices, or as the result of an undesired body state, e.g. the presence of unwanted adipose tissue or wrinkles. Accordingly, the nature of the sensors will vary in order to enable the assessment of the desired physiological condition.

In order to enable the use of one or more sensors, said sensors may be located in a sensor structure. The sensor structure may contain necessary electrical, mechanical and software elements enabling the obtaining of one or more measurements from one or more body zones encompassing one or more body regions, i.e. the transmission and reception of a sensor signal to and from a measured body region within a body zone.

The sensor structure may also have one or more means for conveying said sensor data to one or more comparator for analysis, i.e. one or more means of transmitting either wirelessly or through direct electrical contact received sensor data to the comparator.

In certain embodiments of the invention, individual sensors and/or the sensing and/or therapeutic structure as a whole may have one or more identifiers. Identifiers may advantageously allow tracking of individual sensors and/or sensing and/or therapeutic structures for the purpose of enabling lot identification in case of failures, recalls, or ensuring appropriate application of sensor type to meet individual patient needs. In yet related embodiments, one or more sensor signals may be utilized to form part or whole of the identifier and/or data encryption methods employed with identifier and/or signals. Such uses of identifiers may be utilized to further match sensor/therapy to patient diagnosis, demographics and/or anthropometric attributes.

Sensors may include but are not limited to optical, acoustic, mechanical, electromagnetic, chemical, or electrical means to obtain the physiological, environmental or therapeutic data. In general forms of the present invention, sensors may interact or detect one or more properties in order to enable the determination of one or more regions to receive subsequent therapy. These properties may include those associated with body tissues, fluids or structures, properties of introduced agents or devices, properties associated with the response of the body to such introduced materials, properties associated with undesired materials or organisms, e.g. bacteria, fungi or biofilms, and/or properties associated with the body's reaction to undesired materials or organisms, and/or environmental elements (ambient temp, humidity), and/or anthropometric data.

In preferred embodiments of the present invention, optical sensors may be comprised of at least one photonic energy source enabling delivery of photonic light to a body zone or portion thereof and a corresponding photodetector so configured as to be responsive to resultant signal arising from the interaction of the photonic energy with the body zone.

In general form, photonic sources for use with optical sensors may be comprised of emissive devices or materials including, but not limited to, light emitting diodes (LEDs), organic light emitting diodes (OLEDs), lasers, plasma light sources, incandescent sources, fluorescent sources, phosphorescent light sources, polarized light sources, filtered light sources, or ambient light sources transmitted to inspected body regions by lens, transparent structures or by other means. Likewise, photodetectors for analysis of one or more parameters associated with said photonic sources may be comprised of photosensitive diodes, avalanche photodiodes, or other photosensitive structures, e.g. photographic films or gels, suitable for responding to one or more light energies associated with parameter detection and/or quantification. In other embodiments, the photodetectors may employ filters that are either fixed, e.g. static, or dynamically filtered to requisite frequencies. In addition, structures such as wavelength filters, wave guides, diffraction gratings, amplifiers, etc., may be incorporated into the sensing structure to improve overall photonic sensing capabilities.

Examples of one form of such sensors are optical sensors made from OLEDs and printed photodetectors utilizing wavelengths enabling the determination of body states such as the increased blood perfusion or tissue oxygenation associated with the body's response to the presence of infection. In such embodiments, the sensors may entail a plurality of light sources and photodetectors for the employment of multiple light frequencies in order to obtain the desired bioparameter data. Such sensors may also take the form of sensors sensitive to light scattering or absorption. In still other embodiments, optical sensors may consist of one or more charged coupled devices (CCD) or elements disposed on a sensor structure as to enable inspection of one or more body regions and/or materials derived from said body regions using illumination arising from one or more ambient light sources not directly contained within the structure of the device of the invention.

In general terms, photonic sensors may employ the absorption of selected wavelengths of light by chromophores in the body zone to enable a determination of the status in one or more body regions. Such chromophores may include species absorbing in the ultraviolet, visible or infrared regions of the spectrum. Well known chromophores naturally occurring within mammalian tissues include nucleic acids, hemoglobin, myoglobin, cytochromes, flavins and nicotinamide/adenine containing enzymes. As many of these are related to oxygen content, e.g. hemoglobin or myoglobin, or cell energy status, e.g. cytochromes, measurement of these species may provide useful insight into tissue status within a measured region. Likewise, selected metabolites, e.g. glucose, have signatures to specific spectral range that are useful for their determination and the relative abundance of these species may aid in the determination of tissue region status.

In yet other forms, the optical signals may employ one or more light wavelengths and photodetectors to enable detection of one or more inherent photonic characteristics, e.g. fluorescence, of one or more biological compounds, introduced materials, compounds, or structures, and/or infectious agents, in order to assess body region status. In still yet other forms, sensors may detect photonic emissions associated with activation of one or introduced structures, materials or gene products, e.g. use of genetic marker systems such as a green fluorescent protein marker system activated or turned on by one or more physiological processes.

In still other embodiments, useful information regarding tissue status may be gathered by indirect assessment of light absorbance or scattering within a region. By example, introduced light source may be scattered by tissue components e.g. cells, extracellular proteins, etc. This causes a diffusion arising from scattering of the light through the tissue region. The extent of this scattering therefore reflects in some measure the underlying tissue composition giving rise to the scatter. By measurement of the light intensity and range of scatter at one or more photodetector from one or more introduced light source, useful information regarding tissue structure may then be gained. In general terms, photonic sources and corresponding photonic detectors may be spatially segregated by distances generally of a few millimeters to several centimeters in order to enable passage of photonic signals through volumes of body tissues enabling assessment of one or more desired physiological parameters, e.g. tissue oxygenation or tissue structure. However, other spacings and separations are conceivable, e.g. transmission substantially through a body region and therefore the scope of the present invention is not limited to any one distance, size or spacing of photonic sources or detectors.

To facilitate the introduction of one or more light sources into a body region, gels or other aids may be used that are made of materials that reduce the difference in refractive index between the light source and the receiving tissue boundary, e.g. the skin. Use of such aids may result in a higher efficiency of light penetration into the tissues and thereby improve overall device performance.

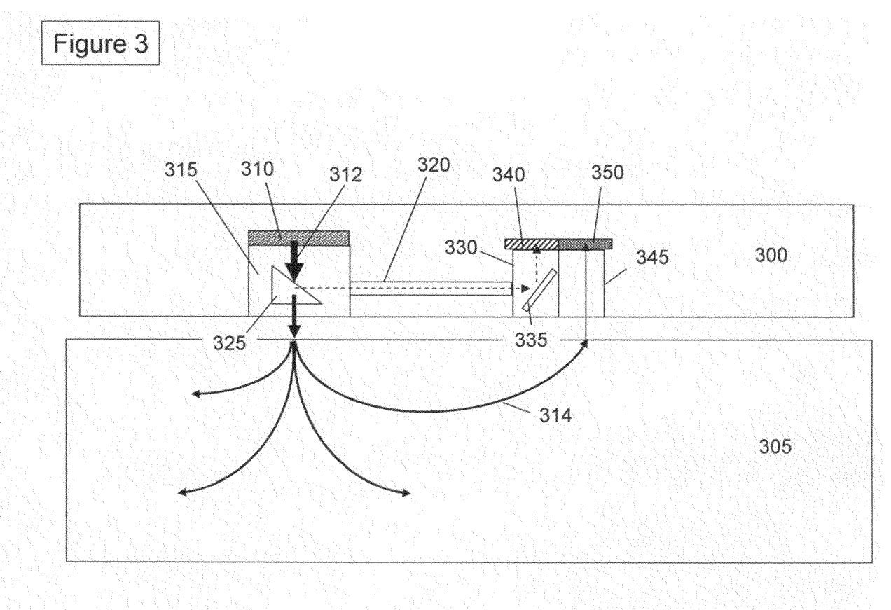

In yet other embodiments, one or more structures may be provided to enable calibration of supplied photonic energies and/or photodetector sensitivity. One example for achieving such calibration function, FIG. 3, is to provide an optical conduit, e.g. a light pipe or wave guide 320, between one or more optical sources 310 and photodetectors 340. In such embodiments, a portion of the emitted light 312 may be selectively segregated by partial refraction using a beam splitter 325 present in structure 315 then the transmitted by light pipe 320 and reflected by mirror structure 335 to calibration photodetector 340 without passage through a body region. Resultant light signal 314 from passage through body region 305 then may be received by photodetector 350 associated with structure 345 and analyzed such that the incident light intensity prior to passage through a body region is known. Related forms of structures and methods may also be utilized in forms of phototherapy delivery to enable calibration of delivered light to one or more body regions and then to measurement photodetector 350.

In a preferred form of the invention, a sensor structure having an overall flexible planar structure having multiple light sources and photodetectors may be employed to conduct photonic measurements over a body zone. In certain instances, optical filters and/or selective wavelengths may be employed to facilitate such measurements. In related embodiments, ambient light or broad spectrum light sources may be employed which are then selectively filtered, e.g. through the use of one or more bandpass filters or high/low cutoff filters, to aid in the delivery and/or measurement of desired wavelengths. In yet other instances, useful signals may be obtained from one or more fluorescent or phosphorescent agents.

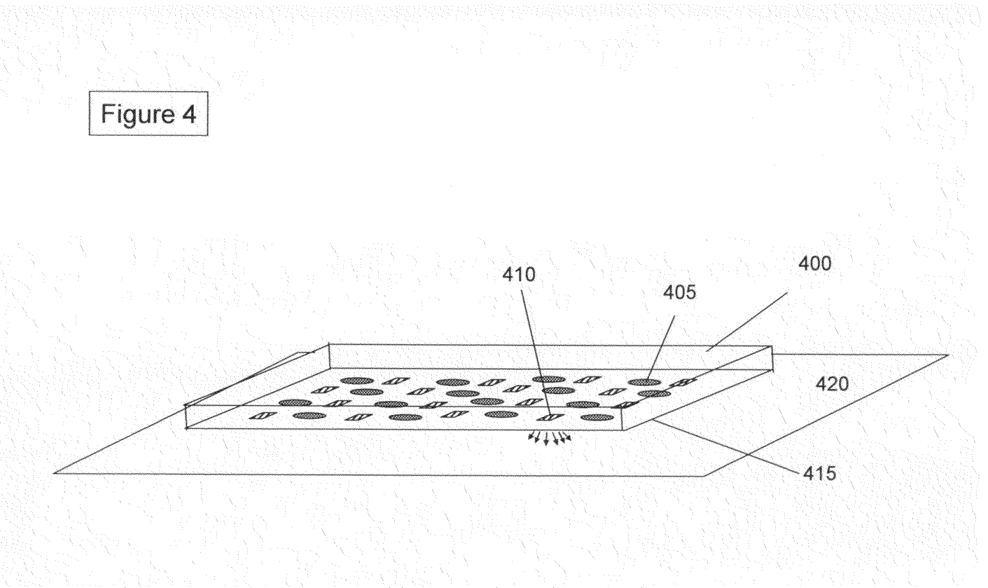

In a preferred embodiment of the present invention, a plurality of OLEDs are employed in a planar fashion upon a first surface of a measurement structure. An example of one possible arrangement of sensors within a first surface is shown in FIG. 4. Emitted light from a single OLED 310 is projected from a first surface 415 of said structure 400 towards a body zone 420 to be inspected. A plurality of photodetectors 405 is positioned on the measurement structure such that photonic energies reflected from one or more body regions is received by one or more photodetectors. In these preferred embodiments, the photodetectors and light sources may be in a substantially repetitive arrangement on a first surface of the measurement structure. By utilizing forms of measurement structures wherein the light sources and photodetectors are arranged in a recurrent pattern on a first surface, multiple body regions encompassed by the body zone overlain by the measurement structure may be readily measured and the responses from each body region subsequently analyzed by the comparator.

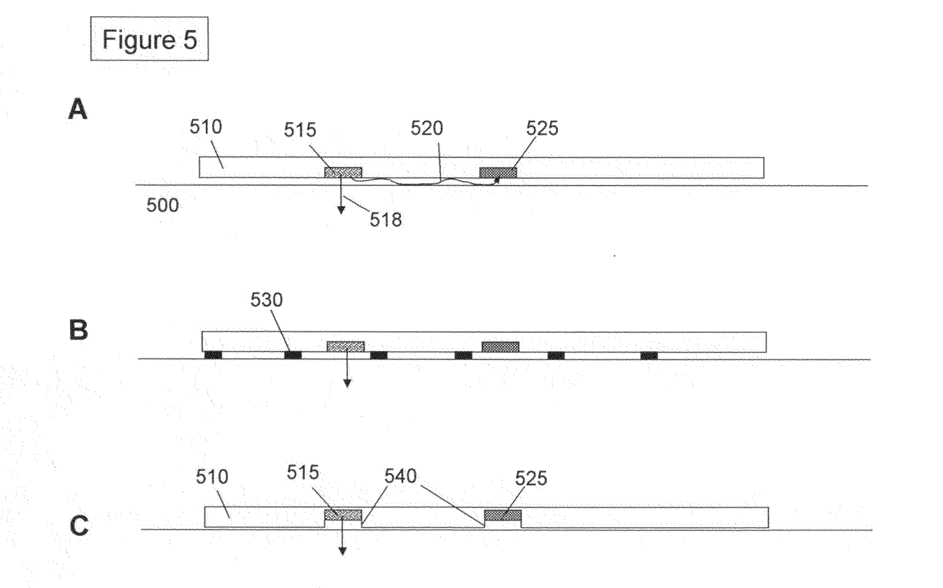

One potential source of loss of signal utilizing a substantially planar arrangement of sensors and light sources is illustrated in FIG. 5A. As shown, one or more light sources 515 is in a known proximity to one or more photodetectors 525 in structure 510 arranged on body zone 500. Light arising from the light source 515 may transit directly 520 to one or more photodetectors without substantially passing 518 into or through body regions of interest. In order to avoid loss of signal and/or signal analysis errors introduced by such undesired photonic pathways, one or more intervening structures, e.g. opaque structures 530—FIG. 5B, may be interposed between photonic sources and detectors. Such structures may consist of ridges or raised areas on the surface such that upon application of the measurement structure to the tissue, an effectively light-proof barrier is established along non-desired lightpaths, e.g. along the surface of a skin or tissue region to be measured. Such barriers may also have the advantage of reducing the impact of stray light upon measurements, such as ambient or other light infiltrating at the edges of the sensor structure. These barriers may consist of opaque materials or materials with dimensions and compositions enabling the absorbing, guiding or reflecting of undesired light away from undesired optical pathways.

Conversely, in alternate embodiments, the light sources and/or photodetector elements may be slightly recessed 540 into the first surface 510 such that the first surface serves to block light transiting effectively directly between light source(s) 515 and photodetector(s) 525—FIG. 5C. In such embodiments, the recesses may be so constructed as to provide additionally useful guidance of light energies, e.g. permitting only light waves of the proper angular orientation to either be transmitted or received. Such embodiments may be useful for ensuring that received photonic energies have transited a desired body region.

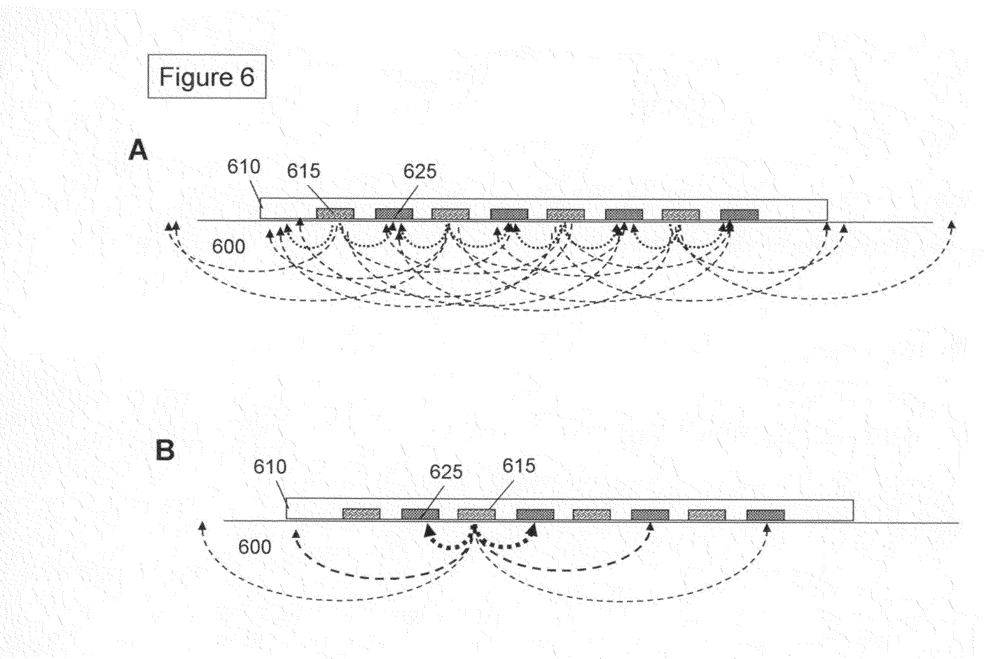

Selective activation of one or more light sources and one or more photodetectors may be also employed to further reduce undesired photonic transits between various sources and detectors as well as enable more defined analysis of individual body regions. Consider a matrix of light sources interposed with photodetectors such as that presented in FIG. 6A. As shown, upon illumination of multiple light sources 615 simultaneously, light received at photodetectors 625 represents a sum total of all tissues transited in body zone 600 and not just a single body region.

In remedy of the aforementioned complexity of signal analysis arising from simultaneous illumination of multiple light sources, light sources, e.g. 615—FIG. 6B, may be sequentially activated thereby differentially illuminating selected body regions within body zone 600 to enable more defined signal analysis pertaining to respective individual body regions. This sequential activation against a substrate with defined optical properties representative of body zone 600 also provides a way to calibrate the device light intensity, photodetector sensitivity and spacing variation upon application to the body, during manufacture, or prior to application to the body. Variation in light source wavelength/intensity as well in photodetector sensitivity is inherent in all manufacturing processes. An offset/calibration factor can be created dynamically or during manufacture and stored to normalized the light output, photodetector sensitivity and relative spacing of these elements for use in subsequent analysis.

That is, signal simultaneously received by one or more photodetectors 625 will be directly attributable to specifically activated light source(s) and therefore may be useful in the calibration of light source and detector and/or improve parameter analysis as compared to signals simultaneously generated from a plurality of light sources. A somewhat related embodiment is conceivable where selective activation of light source(s) may be detected by individual photodetectors such that different tissue depths and body regions may be inspected by selective activation and/or detection of light sources. Various configurations and activation arrangements, including simultaneous activation of widely spaced light sources and detectors within the measurement structure are readily conceivable and the scope of the present invention is not limited to the examples presented above.

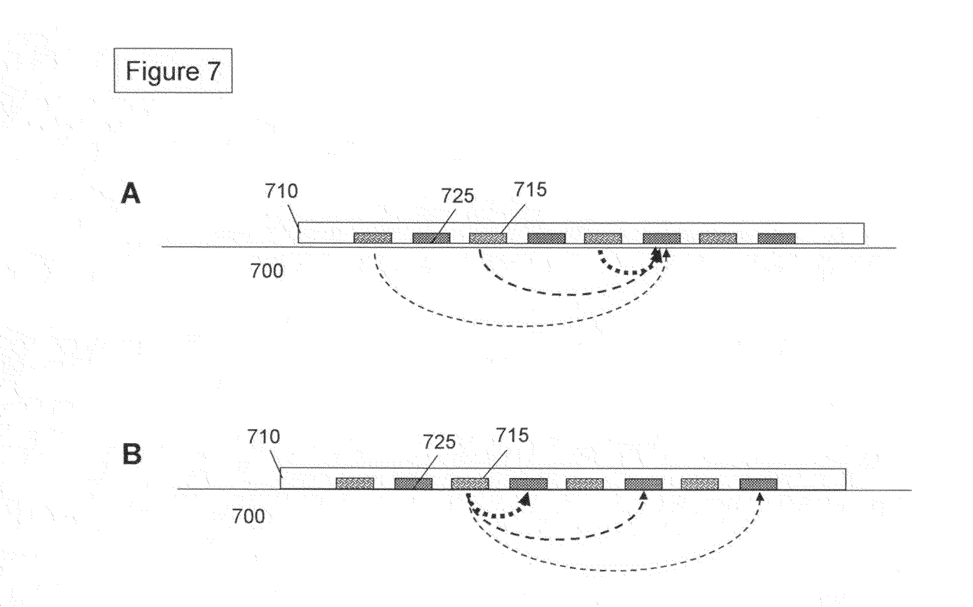

As example use of photonic sensors integrated into a device of the present invention, a plurality of light sources emitting light in the red as well as a plurality of sources emitting in near infrared regions of the light spectrum may be employed to estimate the amount and/or relative percentages of oxygenated hemoglobin or myoglobin in tissue regions. Such measurements may be useful indices of the occurrence of inflammation in such regions. FIG. 7A illustrates one embodiment to accomplish such measurements. As shown in FIG. 7A, light sources 715, comprising both red and infrared emissive sources and photodetectors 725 may be separated on the measurement structure 710 by a distance selected to ensure desired depth of tissue 700 illumination, e.g. to enable measurements of subsurface structures such as muscle beneath skin. In variations of such measurements, as shown in FIG. 7B, a plurality of photodetectors 725 separated over selected distances from one or more light sources 715 may be employed to provide useful bioparameter information, such as oxygen content, within different tissues layers or depths of tissues 700.

In certain embodiments of the present invention, as either part or the whole of the sensing measurement, one or more photonically measured signals may be utilized to ascertain the status of one or more bioparameters in one or more body regions encompassed within a body zone. Such bioparameters may include, but are not limited to, tissue structure, tissue composition or tissue health status as well as measurements related to the presence or absence of one or more disease causing agents including bacteria, fungi and/or structures such as biofilms associated with the presence of these non-desired microbes.

In other embodiments, photonic sensor measurements may take advantage of agents, compounds, dyes, genetically modified biomolecules and viruses, nanostructures, biomolecules e.g. proteins, porphyrins, antibodies, or hormones, or other materials to aid the analysis and identification of body regions that may benefit from therapy. In general forms, the agent may consist of a binding moiety and a visualization structure. The binding moiety may consist of a structure such as an antibody to enable targeting of specific biological structures, e.g. selected bacterial species. Attached to the binding moiety may be a visualization structure, e.g. a quantum dot, a reflective or scattering element, a fluorescent dye or structures/molecules enabling visualization, e.g. enzyme linked signal amplification. In other embodiments, a specific binding moiety is not present within the agent and differential signal accumulation in measured regions is achieved by other means, e.g. directed application, selective uptake by certain cell types, etc.

In related embodiments, the photonic identification agents may be the same as the agents employed in subsequent photonic therapy. Measurements employing such agents may be utilized for the purpose of discerning therapeutic agent concentration and thereby enabling differential adjustment of phototherapy to the measured concentration of agent observed in each region. In order to avoid full activation of such agents as part of the measurement process, one or more techniques such as a reduced intensity, duration or alternative wavelength of measurement illumination may be employed. In a somewhat related embodiment, one or more tracking dyes or materials may be mixed in at a predetermined ratio with a desired phototherapy agent in solution or suspension such that the tracking agent is measured through the use of one or more wavelengths suitable for detection of the tracking agent while having lesser activity in activating the phototherapy agent. From measurement of the tracking agent, the local concentration of phototherapy agent may then be calculated in measured regions.

According to one embodiment of the method of the invention, a multistage measurement process may be employed in the utilization of identification one or more agents for obtaining useful data relating to one or more desired parameters. As a first step, the agent(s) having a binding functionality may be supplied either systemically or locally (directly) to the body zone of interest and allowed to interact with tissues, structures, materials, biomaterials and/or microorganisms to which the identifying binding moiety/agent is targeted. Following an incubation period, e.g. seconds, minutes or hours, the body zone may be subjected to a step for the selectively removal of unbound identifying agents, e.g. the body zone may be rinsed. This step may be utilized to enhance overall signal to noise ratios of identifying agents within body regions. The body zone may then be illuminated to enable visualization and identification of body regions having retained binding moieties/agents.

Additional forms of sensors alone or in combination with the above described photonic sensors may also be employed within the context of the present invention. These sensors may include acoustic sensors based upon conduction of one or more sound or mechanical waves through body regions and may be utilized for various measurement purposes, e.g. for the detection of fluid and/or compositional changes associated with undesired body conditions. Mechanical sensors such as pressure sensors may likewise be employed. For instance, arrays or matrixes of such sensors may be utilized to sense regional tissue properties such as resilience that is altered by the disease progression and or environment e.g. presence of edema, swelling, high blood pressure, or other undesired body states. Electromagnetic and electrical sensors might utilize the employment of one or more applied signals such as electrical impedance or radiofrequency, e.g. ultra wideband, signals to determine body region(s) that might benefit from application of the therapy, e.g. therapies to reduce swelling or edema. Alternatively, these sensors may receive one or body signals in the inspected regions, e.g. temperature or electromyography (EMG) signals, indicative with body states or conditions that would benefit from therapy.

Still other sensors may analyze body fluid components, e.g. for the presence of analytes, metabolites or other biomolecules, with microstructures arrayed on a first surface to withdraw minute levels of fluid and then analyze these fluids utilizing lab-on-a-chip technologies such that the levels of analyte at any one region may be determined.

In yet other embodiments, the measurement structure may incorporate multiple types of sensors for determination of body region(s) status within the measured body zone. Forms of such sensor combinations may be employed to enable redundancy of sensor parameter determination, e.g. multiple sensors intended for the assessment of tissue fluid status/edema. Alternatively, one or more sensors forms may be employed to measure multiple parameters, e.g. regional blood flow, temperature, and the presence of undesired bacteria. Numerous sensor forms and combinations of sensor forms are conceivable and the scope of the invention is not constrained to the examples presented here.

As noted earlier, a preferred embodiment of the present invention incorporates a plurality of sensors, e.g. light sources and photodetectors, in a repetitive gross geometric arrangement such as a grid such that a body zone may be mapped into respective body regions. In one form, this may be accomplished by a first surface of a measurement structure having a plurality of sensors oriented towards the body tissue directly to the body zone in question. By such orientation, a correspondence between sensor location on the measurement structure and the associated body region in direct opposition to specific sensors may be established. Such arrangements facilitate the mapping of body zones into various body regions to be differentially treated. In other embodiments of the invention, sensors may be organized to yield useful physiological data but not be arranged into a grid or other regular pattern. Such embodiments may have sensors arranged to either provide useful vectors of energy through a body zone enabling distinction of one or more regions within the zone, e.g. peripheral sensors transmitting signals transversely across or through a body zone. In other forms, sensor receiving or transmitting elements may be all or partially patterned in a fractal or other geometries that enable optimized energies and determinations not achieved through other repetitive sensor patterns.

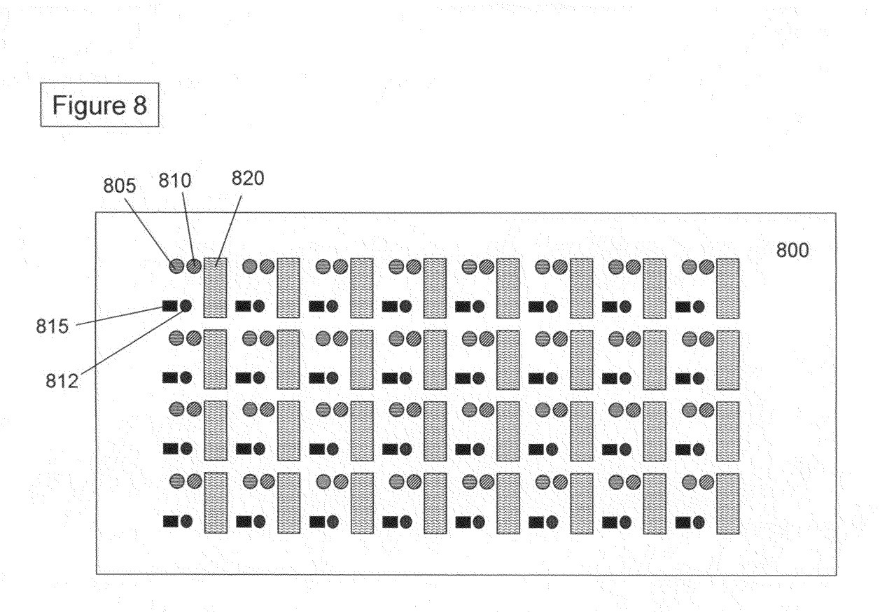

For example, a representation of the first surface of one such structure is shown in FIG. 8. Body of structure 800 contains an array of sensors (light emitting sources 805 and 810 of differing wavelengths, temperature sensor 815 and photodetector 812) so organized as to enable inspection of a body zone when covered by structure 800. Also shown are photonic therapeutic energy sources 820 within same structure 800. Said arrangement of sensor elements and therapy energy sources enables the targeted delivery of photonic energy to one or more regions covered by structure 800.

In one form of this embodiment, the sensors may be comprised of optical sensors sensitive to the presence of blood or blood constituents in the inspected zone and temperature sensors response to surface and subsurface temperatures in the inspected zone. By measurements taken by said sensors, e.g. through sequential activation and recording of data, a map of the underlying tissue status, e.g. healing, infected, biofilm presence etc. may be obtained and inspected for regions having one or more signatures associated with a condition that would benefit from the application of therapy. By way of example, these sensors may be responsive to a condition indicative of localized infection such that altered blood flow such as that associated with inflammation which may occur at the site of the infected region within a wound bed region. Likewise, the temperature of the tissue may be altered at the site of an infection. In still other embodiments, one or more sensors, e.g. bioelectric impedance or ultra wideband radar sensors, may be sensitive to one or more changes associated with local hydration change or edema. Thus, through the use of multiple sensors targeting a common underlying disease or body condition state, the likelihood of false positive and or false negative identifications of body regions may thereby be minimized. In certain embodiments, one or more sensors may also be utilized for therapy delivery, e.g. photonic energy, electrical or radiowave stimulation, etc.

As part of sensor activity, it should be noted that the method and devices of the present invention are not constrained to direct correspondence for the mapping of body regions. Alternative forms, e.g. matrixed electrodes enabling bioelectric impedance measurements, may be employed allowing the reconstruction or mapping of body regions for the determination of the entire body or one or more body regions that would benefit from an applied therapy.

One advantageous use of a patch-like form of the sensor structure is that the location of said body regions may be precisely defined using the structure and location of the sensor arrangement as reference points. That is, by affixing the structure to a body zone, identified body regions may be better located through use of the structure of the device itself for subsequent therapeutic delivery. In the context of the present invention, affixing may include the use of straps or adhesives or through the use of a handheld device held in place for a period of time sufficient for sensor measurement, analysis and targeted therapeutic delivery.

In still other embodiments, one or more fiducial markers may be placed on or in the body to enable subsequent alignment of diagnostic sensing data with prior measurements and or therapeutic delivery activities with identified body regions. Such markers may take the form of reflective inks, RF chips, or passive structures absorbing and or reflecting one or more sensor signals. In selected embodiments, such markers or materials may also be employed to provide calibration to one or more sensor signals.

In yet other embodiments, body measurements, metrics and landmarks may be employed to enable alignment and precise targeting to identified regions with the applied therapy.

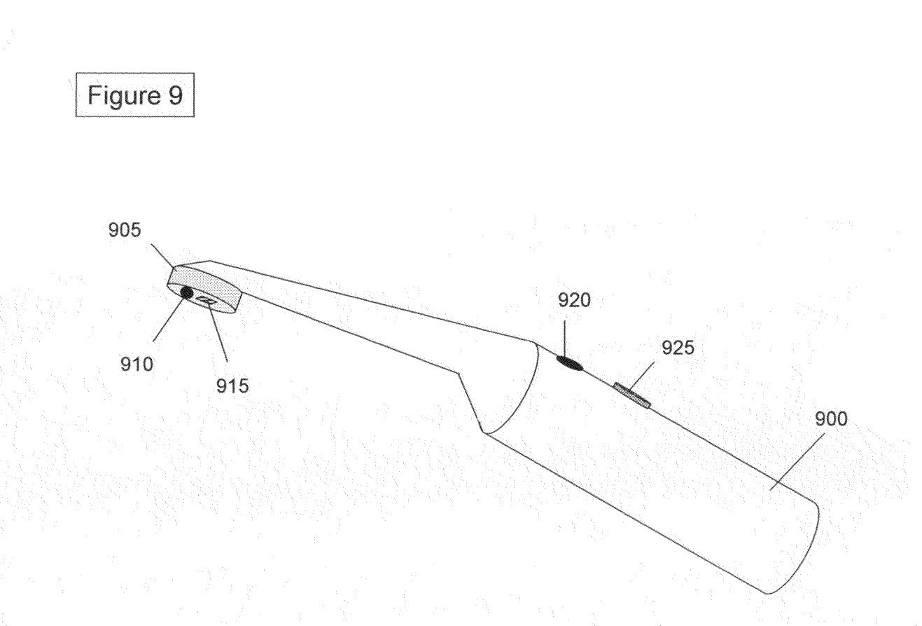

Such use of fiducial markers and/or landmarks may be advantageously employed with those embodiments wherein one or more sensors are moved over a body zone in order to enable the identification of one or more body regions that would benefit from therapy. FIG. 9 presents one such conceptual device wherein the form of the device is as a handheld wand. As shown, located on device body 900 is a sensor tip 905 having sensor 910 and photonic energy source 915. Also shown is an activation and/or on/off switch 925 enabling use of the structure and alert light 920 indicating the presence of a body region that would benefit from therapy. In one form of use, the structure would be traversed over a body zone of interest. In response to detection of a body region, the location of the sensor tip may be registered relative to one or more fiducial points and/or activate the alert light 920.

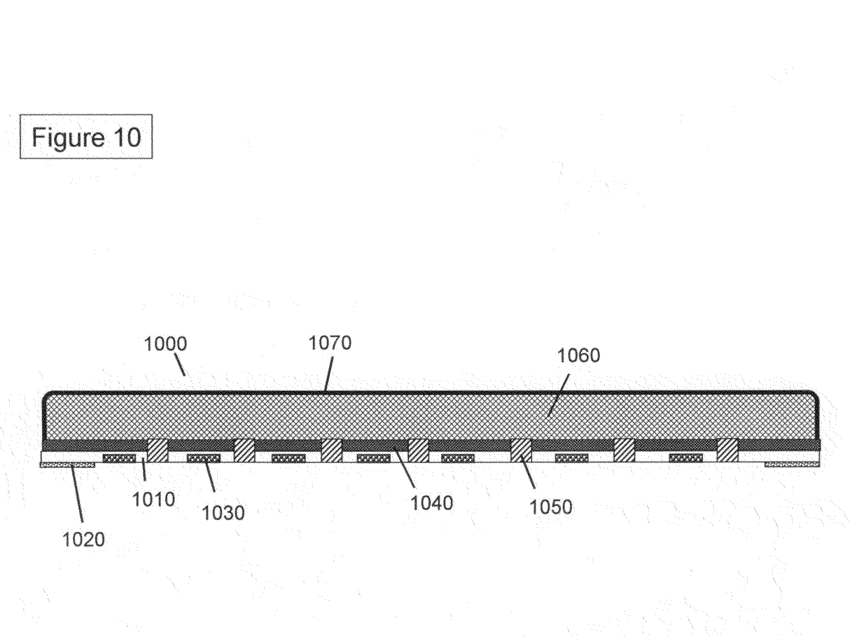

In yet other forms of the invention, additional structures or features may be incorporated into the first surface to enable additional functionality of the device of the present invention. One such embodiment is shown in FIG. 10 representing a cross sectional view of essentially planar device 1000. As shown first surface 1010 has adhesive 1020 positioned on the margin to enable adherence of the first surface to a body zone. Photonic elements (emission sources and photodetectors) are indicated by structures 1030. Also shown is electronic circuitry layer 1040 providing electrical control, communication and comparator activities. In order to enable functionality as a wound dressing in addition to use as a monitor/therapy delivery system according to the present invention, absorbent material 1060 is included within overall device 1000 and is covered by covering 1070. To enable absorption of wound exudate, wicking structures 1050 are interposed between photonic elements to enable conveyance of wound exudate from the region of the first surface to absorbent material 1060, through first surface 1010 and electronic circuitry layer 1040. Such wicking structures may be composed of materials able to enable capillary transport of fluids. In alternate embodiments, these wicking structures may be active pumps, e.g. vias lined with electroactive polymers enabling pulsatile movement of fluid in a directional fashion.

In yet other embodiments, the first surface and sensors may be incorporated into a structure enabling additional medical uses, e.g. prosthetic attachments. In still other embodiments two or more sensors spatially segregated on a body zone may be employed to enable temporal measurements of a physiological parameter between these sensors. Such temporal measurements may include blood pressure wave transduction or nerve signal transduction and thereby enable determination of one or more physiological conditions directly associated with the measurement, e.g. carpal tunnel syndrome in the nerve conduction signals, or indirectly associated, e.g. heart contraction performance.

Additional forms and structures for the sensors of the present invention are conceivable within the scope of the present invention and the invention is not limited to those examples presented herein.

Comparator

The comparator analyzes data received from one or more sensors and/or stored prior measurement data, and/or calibration data, and/or input demographic, anthropometric and/or other clinical data for the purpose of identifying one or more signals associated with one or more body regions that may benefit from therapy within an inspected body zone and then provides this data/analysis to attending clinicians and/or directly to one or more therapy delivery structures. To enable these functions, the comparator may be comprised of one or more signal processing units, one or more processors, one or more data storage components, one or more power sources, and one or more display and/or communication means. The comparator and/or individual elements of the comparator may also have identifiers enabling the tracking of comparator activity and coordination of such activity with one or more sensing structures and/or therapy deliver structures. In preferred embodiments, the comparator is substantially co-located with the sensor structure as part of a single overall structure and may utilize one or more elements of the sensor structure, e.g. power supply, controlling processor, or memory, to enable comparator activities.

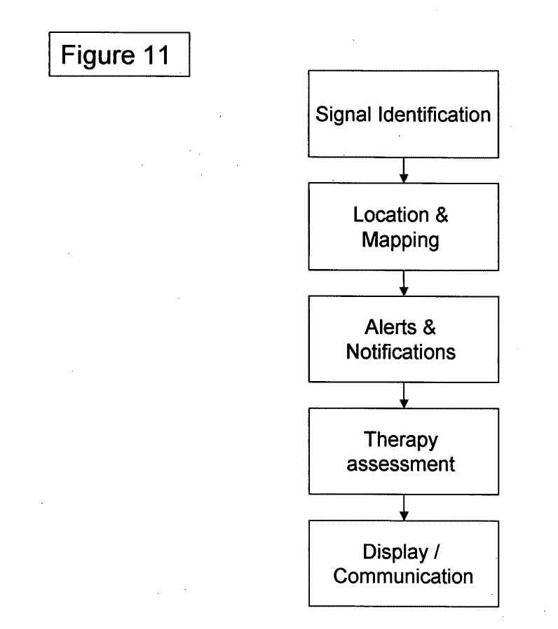

The form and activities of the comparator according to the method of the present invention may take multiple forms or embodiments. For instance, in one embodiment, the comparator may directly display sensor measurements of measured body regions. In a preferred embodiment, illustrated in FIG. 11, the comparator activities may be comprised of multiple functions and activities: As shown, a first function may be to conduct signal analysis of data from one or more sensors corresponding to body regions such that the data is compared to previously determined values, trends or rates of change to enable the classification of the inspected body region as possibly benefiting from therapeutic intervention. A second function of the comparator may be to register the location of said identified regions as they are identified and a third function of the comparator may be to provide one or more notification methods to alert a user of the device that one or more regions has been detected and the location identified. A fourth function of the comparator may be to quantify the extent of the condition identified within a region to enable possible titration or adjustment of the delivered therapy. A fifth function of the comparator may be to provide instructions to the therapy delivery component of the device enabling targeted delivery of one or more therapies to one or more identified regions. Each of these functions is described in greater detail below but it should be noted that within the context of the present invention other comparator functions, activities and order of the functions are conceivable and the invention is not limited to these functions or the order of these functions presented here.

Signal Identification Function One function of the comparator is the identification of body regions that may benefit from therapy using measured sensor data. Comparator signal identification may utilize raw sensor signal data or mathematical transforms of received signal data. Such transforms may include the use of combinations of one or more data sets from one or more sensors including use of data arising from one or more sensors measuring one or more parameters, e.g. temperature sensors for determination of body region temperatures associated with inflammation combined with optical sensors for fluorescent signals indicative of the concentration of a phototherapeutic agent. In addition, body region identification may take advantage of additional input or otherwise provided information indicative of a body region that would benefit from therapy. Such additional input may arise from direct, e.g. clinician, input or from one or more outside databases providing trend, prior measurements and/or data analysis from other individuals having similar conditions and/or therapy needs. Such additional data may include, but is not limited to, demographic data, anthropometric data, environmental data, images, co-morbidities, or other treatment data.

In certain embodiments, additional signals arising from one or more sensors not located on the device of this invention may be included in comparator activity in order to accomplish the identification. Such additional signals may include heart rate, core body temperature, activity levels, etc. In yet other embodiments, additional factors or data may be inputted into the comparator to enable determination. Such additional factors may include anthropomorphic data such as age, weight, height, gender, clinical data such as type of disease, co-morbidities, disease stage, or population/group based data providing feedback from related populations.

In still other embodiments, the comparator may learn or compare over time trends in body zone responses and/or sensor data in order to better define body regions. In related embodiments, one or more aspects of signal data is utilized to modify or correct signal data. Such corrections may include corrections for shifts in sensors performance over time, corrections for errors in device misplacement over repeated uses, and corrections for motion or ambulatory noise. Forms of these corrections may include, but are not limited to, signal averaging, normalization of signal values, filtering or discarding of outlying sensor data, and/or weighting of signal data based upon magnitude of the signal value relative to known accuracy of sensors and/or parameter being measured.

In yet other embodiments, one or more regions and/or identified sections within the body zone may be utilized as internal standards for the calibration for measurements arising from one or more body regions. For example, prior to affixing a sensing/comparator structure over a wound region, a clinician may note that a region outside of a wound is normal in appearance yet the interior of the wound may be classified as a severely inflamed. Such subjective assessments may be translated into a scale, e.g. a numeric scale wherein a score of 1=normal; 10=severely inflamed. Such scales may be based on existing classification methods or may be newly created to better adapt to individual patient conditions. The clinician may then identify the regions corresponding to these ascertained tissue standards and provide the comparator with these scores associated with these regions such that the system of the invention may automatically score the other body regions based upon this calibration. In a related embodiment, one or more calibration standards, materials or devices may be position within the measured zone such that these may be utilized for the calibration of sensor measurements.

In determining whether a signal is representative of a region that may benefit from therapy, use may be made of tabular or other forms of organized data having preset limits, cutoff points and/or relationships to one or more therapies. Such limits and relationships may automatically available/preset or inputted by attending clinicians, etc. These tables may enable discrimination of signals indicative of body regions that would benefit, as compared to those that would not, to facilitate location of said body regions. These tables and/or formulas may have been previously established through empirical observations using these sensor functions relative to physiological outcomes. In addition, such determinations may also indicate the relative need or extent of such therapies as well as the form(s) of therapy to be applied. For example, determination of a high degree of inflammation as revealed by sensor measurements such as those for blood flow or temperature may then automatically be scaled as such and the result used for identification of a therapy such as phototherapy plus anti-inflammatory drug administration to be recommended or initiated as part of subsequent therapy treatment for this particular region as compared to a lesser therapy. In addition, other factors, e.g. patient age, gender, co-morbidities, allergies, general health status, available therapeutic agents, body zone location, etc., may be included in the recommendation of therapy to be applied to the identified region.

Location & Mapping Function A next function of the comparator enables the determination of the location of one or more body regions within the larger scanned or imaged body zone such that therapy may be specifically targeted to these regions. This functionality is relates to the aforementioned identification of body regions that may benefit from therapy by the coordination or mapping of such regions within the larger body zone. In preferred embodiments, the body zone is automatically divided into sections, e.g. individual measured body regions based upon the sensor array matrix design employing the smallest sensor unit that is repeated within the matrix. Each section within the zone then may receive an individual comparator assessment regarding measured status, need for therapy and/or recommended therapy are assigned. In other embodiments, the measured regions are not presented as a fixed grid or location but as general areas representative of degree, extent and types of therapies to be applied.

In other embodiments, the regions within the body zone may be automatically created through self adjustment by the comparator. For example, regions may be defined through analysis of overlapping sensor measurements to create a composite determination of regions within the measured zone that may benefit from therapy. Such regions may not reflect a structure or pattern of any one form of sensor, instead reflecting a sum determination of multiple sensor values and/or inputs.

In still other embodiments, one or more regions within a body zone may be identified as normal which then may be used by the comparator for comparison to other regions within the zone to define one or more of these other regions as normal or as regions that may possible benefit from therapy. Such normal regions may be identified as normal either through inputted values, inputted commands or by comparator activity utilizing either preset values or determinations based upon population algorithms and/or body location.

In yet other embodiments, determination of locations of body regions that may benefit from therapy may utilize tracking or motion data obtained from the sensor structure. The use of such data would be especially advantageous in those forms of the invention where the sensor structure is a wand or other movable structure. In still other embodiments, the location correspondence is transitory in nature, and may be directly linked to signaling mechanisms to indicate the positioning of one or more sensors over a body region that would benefit from therapy.

Accordingly, the location function may incorporate grid or other coordinate mapping tables associated with the structure of the sensor structure. Alternatively, this mapping may be reference to dimensions, structures or other aspects relating to the measured body zone or regions comprising the zone. In related embodiments, this functionality may take advantage of introduced or naturally present locators or fiducial marks to provide orientation to one or more signals indicative of a body region that would benefit from therapy. The relative locations of these identified regions and corresponding sensor data as well as possible recommended therapies/degrees of therapies may then be stored by the comparator for subsequent alerts, display and/or therapeutic activities.

Alert & Notification Function As a next function of the comparator, an alert, a noise or other form of notification may be emitted or displayed to indicate that the comparator has assessed measured sensor data and associated body regions for possible therapeutic treatment may be employed, e.g. that the sensors measurements have been successfully (or unsuccessfully) completed and/or one or more regions have been identified that may benefit from therapy. Such alerts or notifications may take multiple forms including those forms associated with automated use of the device of the present invention. In other embodiments, the alerts may be representative signals associated with the body region and provided during use of a sensor structure consisting of a movable structure such as a wand. In alternate forms of the invention, alerts may be comprised of event-type notification to the clinician arising from a change in measured status during periodic or continual monitoring of body zones.

Forms of alerts may include flashing lights, an auditory signal or tone or vibration of the device. In alternate forms of the invention, the alerts/notification take the form of alerts presented on a display or visual map of a body zone indicating one or body regions identified as benefiting from therapy. In certain embodiments of the invention utilizing movable sensors, the sensor structure may be repeatedly passed over a body zone such that upon encountering one or more body regions that are identified as possibly benefiting from therapeutic treatment, an alert such as a blinking light or other form of notification may be made.

In still other forms of the invention, the alerts may be transmitted to one or more remote data receivers, e.g. through wireless connection, to enable notification of clinicians or other third parties of the presence of body regions that would benefit from therapeutic treatment. Such notifications may also include relative forms of therapy to be applied to one or more identified body regions.

Therapy Assessment Function As a next function of the comparator, assessment of therapy(s) to be delivered to individual body regions within the body zone may be compiled for use in subsequent therapy application to the respective body regions. In addition, the therapy to be applied may be possibly optimized across these regions. Such optimization may include interpolation of therapy deliver, e.g. photonic intensity or duration, based upon spatial arrangement of body regions to receive said therapy such that a smooth transition of therapy is administered to two or more adjacent body regions. For example, consider a scenario wherein two adjacent body regions have been evaluated and one has been determined to require intensive phototherapy and the other does not require therapy. In such instances, to ensure that the region requiring intensive therapy fully receives the recommended therapy, the adjacent region may receive a partial amount of therapy to compensate for any loss of effectiveness of therapy delivery due to edge effects (loss of photonic intensity) arising from the transition from one region to the other. Alternatively, such optimization may take advantage of preset rules or external guidance to adjust therapy to accommodate overall therapeutic intent. Such external guidance may include wireless communication with one or more remote data management systems for input possibly involving determinations of current clinical practice to such regions by external authorities, automated optimal therapy based upon population-based responses, etc.

In certain embodiments of the present invention, such compilations may be linear representations of one or more data sets received from one or more sensors. In other embodiments, such compilations may be presented as relative to corresponding body regions not benefiting; e.g. percentage or ratiometric presentations of comparative data sets. In related embodiments, the compilation may include trend analysis over time involving a plurality of measurements taken at multiple time points. In still other embodiments, such quantification may include determination of body region areas and change in body regions over time. Such representations of quantified signals may also include predictions or projected values that occurred either before the initiation of therapy or are anticipated to reflect the project response of one or more body regions or the body zone receiving therapy.

Display & Communication Function A next function of the comparator may be to provide information regarding the status of the measured body region and subsequently derived information to patients, clinicians and/or one or more therapy delivery structures. In certain embodiments, said information may also include information regarding possible therapy to be administered and/or treatment regimen, e.g. application period, frequency and/or strength, to one or more body regions.

As one form of display of measured data and/or quantification of one or more region(s) within a measured body zone, embodiments of the invention having a planar measurement structure on a first surface may have a planar display mounted on a second surface. In such forms, the display on the second surface may have direct correspondence to body regions measured utilizing a first surface having a plurality of sensor elements, e.g. a matrix of printable photonic energy sources such as OLEDs and corresponding photodetectors. Such planar, controllable display may be accomplished through the employment of visual display elements, e.g. OLED-display images, controlled by display control circuitry of the comparator. One such form of such a display may be in the form of a matrix of separate elements which displays measured status/images of the underlying body regions and/or numeric values or suggested therapies associated with each region. Advantageous use of printed OLED photonic sources and photodetectors as well as select printed circuitry elements, e.g. transistors, for sensors and display circuitry, may enable an overall planar and flexible measurement/comparator with display with minimal thickness between the first and second surfaces.

In selected embodiments, said second surface display may not be activated until the reception of a signal, e.g. a wired or wireless transmission of an energy from an activating system that results in the turn on of the display functionality for a period of time, e.g. a few minutes. Such functionalities may be advantageous for the conservation of battery power to extend useful platform lifetime. In other forms of the invention, alternate means of activating the display may be employed, e.g. pressure switches and/or light activated sensors responsive to certain frequencies of light present on the second surface which then switch on the display.

In further forms of such embodiments, the display functionality and the sensing functionality may comprise a “smart window” of the underlying body zone, which provides an image representative of the tissue covered by the sensor/comparator structure. Additional details regarding the status of regions within the larger body zone image may be included into such representative images. Such details may including the highlighting of tissue regions that might benefit from phototherapy, numeric overlays of data indicative of the status of various body regions, or display of images representative of prior status such that tissue status over time. In still other embodiments, such “smart windows” may also receive and display prognostic advice, therapy recommendations, etc. for individual body regions and/or the physiological state as a whole from one or more communications to the comparator from one or more remote data management systems. In preferred embodiments such communications are made by wireless means such that the comparator structure remains unencumbered by wires or other such structures to enable remote communication.

As a further embodiment, the second surface display may contain one or more touch sensitive regions, e.g. printed capacitive elements, such that additional device functions or information, e.g. images, of the measured regions may be brought into view at one or more regions of the display. In such instances, a display having multiple regions presented may then enable more detailed display of one or more regions, e.g. a zoom imaging functionality, based upon touch activation of the displayed region(s) of interest. In related embodiments, such touch sensitive elements may be also utilized to initiate therapy treatment in one or more identified body regions.

Communication of comparator analysis to the therapeutic delivery structure may be through direct linkage between comparator and the therapeutic delivery structure. Such communication is readily envisaged in embodiments of the device wherein the sensor, comparator and therapeutic structures are physically contained within one overall structure. In other alternate embodiments, the communication may be through one or more wired or wireless transmissions, e.g. wireless via electro-magnetic, acoustic or optical signal transmissions. In such embodiments, multiple forms of sensors may be utilized with multiple forms of therapy delivery structures through one or more comparators in order to provide the beneficial delivery of therapy to one or more regions.

Numerous forms of comparators, displays and communication of data are possible and the present invention is not constrained to those examples and functions and/or sequence of functions of the comparator presented herein.

Therapy

Upon receipt of information or directions originating from the comparator regarding the identification and location of one or more body regions that might benefit from therapy, the therapeutic structure may deliver one or more therapies to one or more identified body regions. In general forms of the present invention, one or more therapies may be tailored to one or more body regions based upon comparator analysis. Such tailoring may include regions to be addressed, type, quantity and pattern of therapy to be delivered as well as the delivery period. In further embodiments of the invention, measured body regions may have individual identifiers associated with each region such that individual therapies to each region may be more readily assigned and tracked for efficacy. Such identifiers may be based in part upon measured sensor data for said region.

Therapy delivery may be automatically initiated upon receipt of instruction from the comparator or upon command/action by a clinician or other operator. In preferred embodiments of the invention, the therapy delivery structure is substantially co-located with the sensor structure and comparator as part of a single overall structure and may utilize one or more elements of the sensor structure and/or comparator, e.g. sensor energy delivery sources, circuitry power supply, controlling processor, or memory, to enable therapy deliver activities.

In various embodiments of the present invention, therapeutic delivery body regions and/or therapeutic activities may be adjusted through input by a clinician and/or from other diagnostic and/or therapeutic systems. Such adjustment may include the expansion or reduction of the area of the body region identified by the comparator for treatment as well as alteration of therapy paradigm to better address specific needs of the patient, e.g. addressing co-morbidities or other physical impairments. In alternate scenarios, the therapy delivered by the therapeutic structure may be supplemented by other forms of therapies not part of the system of the present invention. Such supplemental therapies may take the form of delivered agents, drugs, nutrition change, or other therapeutic energies, e.g. radiation treatments, and the scope of these additional therapies is not limited to those examples mentioned herein.

In still other embodiments, multiple photonic energies may be delivered simultaneously to enable “two photon” processes or activation. As a further preferred embodiment of the present invention, said photonic energy may interact with one or more introduced light reactive species and, by doing so, initiate a therapeutic activity. In such preferred embodiments, the light reactive therapeutic species may be substantially inactive until irradiation with therapeutic light energy of one or more specific wavelength(s). In such embodiments, the sensing activities and subsequent comparator activities may involve, at least in part, quantization of delivered light reactive species in one or more body regions and adjustment of subsequent therapeutic photonic energy delivered to the quantity of light reactive species identified by the measurement structure.

Light reactive species utilized for the purpose of phototherapies may be chosen from one or more agents responsive to photonic energies, including ultraviolet, visible or infrared light energies. Such agents include, but are not limited to, molecules such as photosensitizers consisting of organic dyes, porphyrins, nanostructures, or organometallic compounds. As a further extension of the present invention, the photosensitizers may include one or more methods enabling enhancement or concentration in body regions and/or cells types or structures of therapeutic interest, e.g. through the use of binding moieties such as antibodies enabling targeting to specific cell types, or through the use of structures such as magnetic nanobeads coupled to the light reactive species to enable one or more external methods of concentration. Charged species such as polycations may also be employed to better enable interaction with cellular structures of opposing charge. One such form of a target photosensitizer is cationic protoporphyrin wherein additional chemical moieties are attached to the photoreactive porphyrin species thereby enhancing the concentration or localization of the photoagent to desired cellular structures. Overall, methods for concentration may prove advantageous by targeting photosensitizers to areas thereby substantially reducing the overall amounts of light reactive species needed.

As a further refinement to certain embodiments, one or more light reactive species, e.g. photosensitizers, may be delivered by systemic methods such as injection or ingestion, or region methods such as selective deposition as creams, sprays or gels, etc., to identified body regions. Such methods may also enable the deliver of light reactive species to a body zone in general and through the methods and system of the present invention, selectively target body regions within this zone for photonic-based therapies without the need to control the application to one or more regions specifically. In yet other forms of the invention, the photosensitizer may be incorporated into a surface of the therapeutic structure which in turn is associated with a medical device such as a catheter or implant. Upon selective activation whether by light or other means, the photosensitizer may be released to one or more targeted body regions and then activated through one or more optical means associated with the device of the present invention. Such release may also include therapy delivery effectively employing passive basal discharge from an eluting material or structure which then upon activation results in an enhanced, e.g. bolus, release of therapeutic agent.

In select forms of the invention, the delivery of a light reactive species may be directly coupled with the delivery of therapeutic photonic energies. Such coupling may be occur within a relative short period of time, e.g. minutes or seconds, such that the light reactive species remains relatively localized to a desired body region. Alternatively, the delivery of the light reactive species may precede the delivery of the photonic energies by a period of time, enabling diffusion of the light reactive species through a larger tissue area and possibly enable concentration within one or more body regions and/or cell types, tissues or biological structures such as biofilms. In still other embodiments, the photoactivation may be substantially pulsatile in nature to better allow replenishment of needed sustaining elements such as nutrients and/or oxygen in the treatment region. By way of explanation, free oxygen may be converted by a photosensitizer to an active species such as singlet oxygen upon irradiation and therefore oxygen concentration in the target region may deplete over time. Enabling time for oxygen from surrounding regions or areas to diffuse into the target region by pulsatile delivery of light energies thereby serves to aid in maintaining maximal efficiency of reactive oxygen species formation. In still other versions of forms of the invention utilizing light reactive species such as photosensitizers, one or more agents such as antibiotics or topical anesthetics may be administered or delivered to identified target regions in conjunction with the phototherapy such that possibly improvement of therapy through synergy of combined treatment regimens occurs or discomfort arising formation of the reactive species, e.g. singlet oxygen, is minimized.

In yet other forms of the invention, the light reactive species may be delivered substantially in advance of phototherapy delivery. In still other forms of the invention, the light reactive species may be part of structures or assemblies introduced into one or more body regions, e.g. as light reactive polymers or coatings on stents or implants capable of releasing drugs or agents upon irradiation.

In other forms of the invention, the photonic energy by itself supports the desired phototherapy without the need for one or more applied light reactive species, e.g. photosensitizers. Such forms of the invention may take advantage of the selective interaction of certain light wavelengths, e.g. ultraviolet, with various body structures such as deoxyribonucleic acids, thereby resulting in a loss of non-desired cellular functions or activities. In alternative forms, the photonic interaction, e.g. red or near infrared irradiation, may result in increased cellular activities and metabolism thereby improving overall tissue healing. In still other forms, the light energy, e.g. infrared, may be converted to other forms of energy, e.g. thermal energy, and thereby provide a therapy in the intended body region through increased local vasodilation resultant from such warmth. In variations of this latter form of energy, such thermal energies may be utilized to selectively disrupt cellular components without major thermal damage to non-intended cell types or regions, e.g. through induced apoptosis of targeted cells through repetitive heating/cooling cycles with limited heating. Such selective targeting may be useful for selective remodeling of body regions, e.g. for wrinkle removal, mucosal tissue regeneration, adipose tissue removal, and post-surgical scar modification, e.g. scar tissue formation mitigation.

In yet other embodiments of the present invention, the photonic therapy initiates the release of one or more therapeutic non-light reactive species, compounds or agents entrained within, linked to or otherwise affixed to one or more photo-labile structures, molecules or polymers. Such materials may take the form of photoreactive linkages whereby irradiation disrupts a photo-labile bond and results in the release of one or more therapeutic agents. In alternate forms of the invention, the light irradiation may initiate the release of one or more therapeutic agents through the activation of a photocell or other photoresponsive element, thereby either directly or indirectly resulting in the release of the agent.

In somewhat related embodiments, one or more targeted therapeutic energies, e.g. photonic or electromagnetic, may be employed to remodel or alter the physical dimensions of either naturally occurring or introduced materials within one or more body regions. Such remodeling may be desired for a variety of conditions, e.g. skin remodeling by one or more photonic energies in the infrared region for the purpose of wrinkle removal, or for implanted prosthesis remodeling by application of photonic energies interacting with photolabile polymers or other photolabile materials comprising a portion of the implant thereby enabling a change in dimension or shape.

In still other forms of the invention, one or more therapeutic agents may be released from storage means located on the therapeutic structure and targeted by selective delivery to one or more identified body regions. Such agents may include antibiotics, therapeutic compounds, nanostructures, naturally occurring substances such as honey or tea tree oil; shown to have medicinal value or enzymatic agents/gene therapy materials. Storage means may include one or more reservoir structures having pumping and/or valving structures, or entrapment within structures, polymers or other devices selectively enabled to release the desired agent upon command.

In yet other embodiments, the therapy may be targeted to tissues or organs not directly within the measured body zone. In such instances, such targeting may lead to one or more body responses, e.g. hormone release, nerve stimulation, etc., that results in a desired therapeutic action at one or more of the body regions.

In general, identifiers associated with sensor structures, comparators and/or therapy delivery structures may be employed. Among other benefits, such identifiers would enable tracking of device component activities and enable logging of component activities relative to the treated individual. In addition, numerous possible embodiments of the present invention are conceivable and the scope of the present invention is not limited to those embodiments presented herein.

### EXAMPLES OF USE

The present invention may be employed for a variety of uses and applications. These applications may include uses as devices or portions of devices located on the skin and/or other exterior surfaces, e.g. oral mucosa. Alternatively, these devices may be implanted on or about targeted tissue regions, medical devices or body organs.

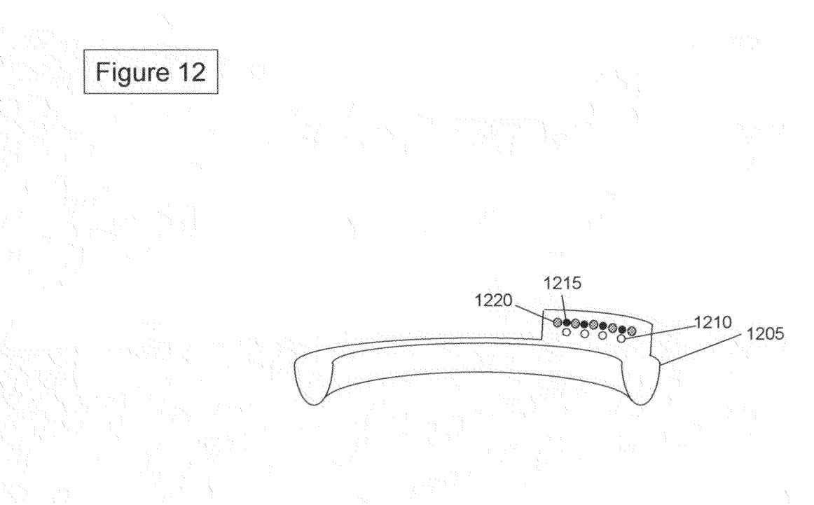

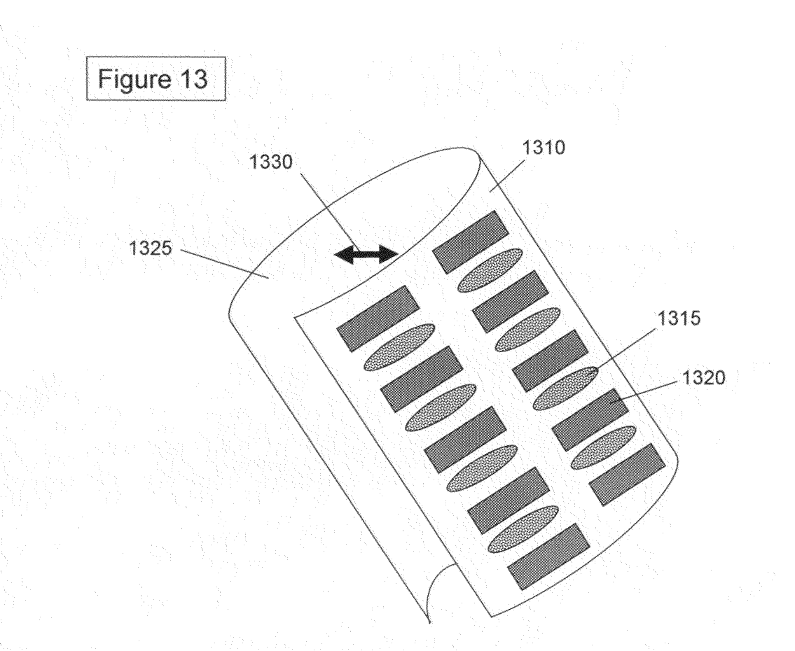

The following examples are intended to serve as general indication of the range of applications to which the method and devices of the present invention may be advantageously employed.
Periodontal disease—The development of periodontal disease is frequently associated with inflammation and other disruptions of normal tissue structure. In one form, a device of the present invention would resemble a mouth guard that would be applied after rinsing the mouth with a photosensitizer and is shown in FIG. 12. In one use, device 1205 with the sensors 1210 and 1220 would inspect at least a portion of the buccal cavity, gums or teeth. Upon detection of one or more lesions, tissue injury, infection, or inflammation, a targeted delivery of photonic energy would be delivered to the region of the lesions, injury, infection, etc., by light sources 1215, thereby sparing adjacent regions from possible harmful effects of the photosensitizer activity. In a further embodiment, the device would be used on a periodic basis, e.g. twice daily, and automatically track lesion, injury, infection, etc. healing, enabling remote assessment of patient status for the clinician. In still other embodiments, the device of the present invention may be incorporated into a bruxing guard and used on a nightly basis.
Wounds—Many forms of wounds would benefit from both accelerated healing and the automatic detection and therapeutic treatment of interrupted healing caused by co-morbidities, tissue dysfunction and/or infection. Still other forms of wounds would benefit from pre-emptive strategies, e.g. treatment prior to full eruption of pressure ulcers or bed sores. In one embodiment of the present invention, the therapeutic healing may be accomplished by the delivery of one or more therapeutic light energies, e.g. that supporting enhanced cell energy production. In addition, the device may also deliver of photonic energies suitable for the reduction of bacterial or other infective agents. Such phototherapy may be accomplished directly through the use of one or more light sources or through the use of one or more photosensitizers in conjunction with one or more light sources to enhance the destruction of non-desired infections and/or body responses, e.g. scar tissue formation.
Biofilms—Biofilms are believed by the NIH to be associated with 90% of all infections. Biofilms may be associated with disease states such as ear infections, periodontal disease, vaginitis, etc. In one form of the present invention, one or more light energies may be employed with the use of one or more photosensitizers to enable disruption of the biofilm matrix and the destruction of the supporting bacterial and/or fungal infection. In such embodiments pulsatile delivery of the phototherapy may be employed to more advantageously allow replenishment of oxygen following depletion by the photosensitizer which converts free oxygen to reactive singlet oxygen and thereby increase the efficacy of the photodynamic therapy. In yet other embodiments, the therapy may be coordinated with other therapies, e.g. washing or mechanical disruption of biofilms through ultrasonic treatment, as part of a therapeutic response.
Athlete's foot—The use of photonic therapy with or without photosensitizers may be used in the treatment of Athlete's foot fungus and related conditions. In such instances, sensors may include the ability to detect the presence of chemicals, liquid or vapor phase, associated with disease presence, e.g. odors, pH changes, etc., and couple this with therapy delivery. In one embodiment, the device of the invention is in the form of a wand wherein the tip contains both sensors and phototherapy delivery means. In such an embodiment, upon detection of an infected region, the comparator might flash a light as an alert and the photonic therapy be automatically delivered at the time of the flash. In a variation, the device might flash a light indicative of the presence of the infection and photonic therapy would be initiated by the user in response by pressing an activating button.
Nail fungus/disease—Nail disease such as fungal infection typically results in discoloration and abnormal growth of the nail. Therefore, in one form of the invention, the sensors may utilize reflected light sources to determine topological or structural change in the nail structure itself and/or the underlying discoloration associated with infection. The therapeutic structure may, in one instance, take the form of a hinged structure to be fitted over the end of the affected digit, enabling prolonged treatment, e.g. multiple minutes, which may be utilized periodically by reapplication of the therapeutic structure. In a preferred form of this invention, the photonic detection sensors are combined with the therapeutic structure and comparator with simplified indicators provided on one or more surfaces to indicate the identification of an infected nail and the initiation/completion of a treatment session.
Acne—The presence of acne results in creation of dysfunctional normal subcutaneous vascularization patterns and therefore, in one embodiment, comparative surface reflection may be utilized to identify body regions that would benefit from treatment. In an alternate embodiment, the sensors may be sensitive to the presence of the concentrations of bacteria and respond to secreted chemical released directly by the bacteria or by the body in response to the presence of bacteria. In one form, the overall structure of the present invention would take the structure of a patch integrating sensors, comparator and therapy delivery. In such form, a photosensitizer may be applied to the skin prior to patch application, enabling automatic targeting of affected regions while avoiding undesired therapy on healthy skin.
Eye diseases—Numerous eye diseases are known to benefit from the use of photosensitizer therapy, including macula degeneration. In such implementations of the present device, the sensor system may be physically separated from the therapy delivery structure but linked through the comparator. In one such embodiment, the comparator automatically identifies the body region (e.g. hypervascularization) of the eye that would benefit from phototherapy through use of comparative mapping algorithms and then enables automatic application of targeted phototherapy when identified region and therapy delivery are in alignment, e.g. when a therapy light source, such as a laser, is identified through another light source as being targeted to the appropriate eye location.
Ears—Infections such as “Swimmer's Ear” are common issues that would benefit from photonic therapy. In one embodiment, the device of the invention approximates an ear plug having sensors, comparator and photonic therapy delivery means. In such embodiments, sensors may respond to altered vascular structures (inflammation) and/or the presence of discharge associated with infection. As one embodiment of therapy for treatment of the condition, the device of the invention also contains additional treatment, e.g. release of one or more antibiotics, in conjunction with the photonic therapy, in order to achieve the intended benefit. A series of ear-plug devices may be prescribed, enabling automatic tracking and therapy delivery over the course of treatment. For ears not requiring treatment, i.e. healed, the sensing function would preclude therapy delivery unless clinician-based commands are instituted.
Nasal conditions—Nasal conditions may range from simple infections to cancerous growths. Accordingly, sensors employed may vary from optical sensors able to sense the presence of inflammation to bioelectric sensors responsive to the change in cell structure associated with abnormal cell growth/proliferation. Phototherapies delivered may differ dependent upon the underlying condition to be treated. In one instance, the treatment may be delivery of a photonic energy to enhance the bodies' own immune response in the desired nasal region. In other instances, the treatment may utilize one or more photosensitizers that, when combined with the targeted delivery of photonic energy, result in destruction of the intended abnormal growth. In one form, the device of the present invention for nasal systems may take the form of a wand having both sensor and therapy delivery located in a tip or structure to be inserted into one or more nasal regions which is then activated by a control button and having an indicator light(s) to indicate device activation and operation.
Upper Respiratory Tract Infections—Frequently related to nasal infections are upper respiratory tract infections which may include the nasal passages but also may involve the sinuses, larynx or pharynx. In such instances, a probe with sensors may be inserted either into a nostril or through the mouth, possibly with the use of one or more local anesthetics to reduce discomfort and regions of inflammation noted. In response to detection of one or more regions of inflammation, one or more therapeutic agents, e.g. an antibiotic spray, may be immediately released or released upon command at that site and to that region through a therapy delivery port or nozzle. Other forms of treatments and/or sensors are conceivable for the detection and treatment of upper respiratory tract infections.
Urinary Tract Infections—Urinary tract infection cause significant discomfort and may be associated with infection of the kidney or bladder. In one form of the invention, sensors may be incorporated into a patch-like structure placed over the bladder on the outer aspect of the body or kidneys. Respective deep tissue zones may be measured for the determination of the presence of infection/inflammation in one or more deep tissue regions. Such determinations may include comparison to surrounding deep tissues as controls. Once identified, one or more therapies, including antibiotics, may be targeted to the appropriate organ and/or tissue region.
Vaginal Infections—Various infections of the vagina, e.g. yeast or bacterial, may be suitable for treatment using the present invention in one or more forms. For instance, the device may take the form of a pill or structure encapsulated in a soft support to be place in the orifice to enable extended functional activity. One such embodiment might be with the sensors responding to fluid composition changes, e.g. pH, viscosity, clarity, odor-related compounds and/or color change, associated with the presence of undesired flora. Upon detection, the device may initiate one or more therapeutic responses including photonic energies intended to eliminate undesired flora and/or the release of one or more sensitizers that upon photoactivation are toxic to undesired flora. In such embodiments, the body zone being inspected may be indistinct from the body region being treated.
Hair loss—Hair loss such as that found in male pattern baldness is a condition attributed in large part by the genetics of the individual. However, topical medications that either increase local blood vessel dilation or disrupt biochemical pathways involved in hair loss are known. In the present invention, sensors may not only be sensitive to the presence or absence of hair but also to the degree and extent of subcutaneous perfusion of blood. In such instances, one embodiment of the invention might stimulate one or more regions deemed by the comparator to have less than optimal levels of skin follicle activity responsible for hair growth. Such photostimulation may take the form of excitatory stimulation, increasing the activity of hair follicles directly or may take the form of stimulation intended to encourage blood flow. In certain circumstances, this latter stimulation may take the form of photonic energies converted to regional heat. The overall form of the device may be in the form of a cap enabling extended placement on the head without the use straps, hooks or adhesives.
Skin conditions—Numerous skin conditions, e.g. psorasis, eczema, or cancer, may benefit from the use of the present invention. The invention may be in the form of a patch having sensors and phototherapy structures incorporated into a single unit able to conform to body shapes. In alternate embodiments, the form of the device may be as a wand or other structure to be moved over one or more body zones. As in other examples, photonic energies delivered as therapies may be directly beneficial, stimulating blood vessel growth and/or healing processes, directly act upon undesired activities including undesired body responses and/or directly attacking viruses or other causal agents. In yet other embodiments, the photonic energies may be directed towards one or more photosensitizers to accomplish one or more therapeutic objectives. In related applications, the method and devices of the present invention may be used for the management of wrinkles, scarring and/or the control of skin cancer through the targeted release of one or more agents. Such devices may take the form of facemasks worn overnight such that the sensing and therapy may be applied during sleeping hours.
Implantable or inserted medical devices—In broad terms, implanted or inserted medical devices may experience contamination leading to biofilms or other infections, and/or require management of the device's integration into the surrounding tissue, e.g. increasing surrounding fibrous growth to increase mechanical stability or reduction of the body's foreign body response, in order to achieve full functionality and therefore may benefit from the present invention. Such medical devices may include, but are not limited to: catheters, catheter sheaths, prosthetic implants, endotracheal tubes, intubation tubes, colostomy tubes, contact lenses, or active devices such as pacemakers, implanted drug delivery systems, implanted glucose sensors, etc. Forms of the device may include those that are effectively separate from the implanted medical device or integrated into the medical device.
Implanted vascular grafts, fisutulas or stents—Related to implanted medical devices, structures such as grafts or stents or surgically connected structures such as arteriovenous fistulas may benefit from forms of the present invention that continuously or periodically monitors regions of the graft or fistula for changes indicative of stenotic lesion formation. Upon detection, one or more agents and/or therapeutic energies, e.g. electrical stimulation, may be targeted to the region(s) of interest. Shown in FIG. 13 is one illustration of this embodiment with sensors 1315 and therapy delivery 1320 effectively traversing body region 1325, e.g. a vascular structure, indicated by arrow 1330 such that individual body region of vascular region may be measured and selectively targeted. In general, this form of the present invention may be combined with other forms of the present invention for the control of biofilms on these structures and/or medical implants.
Orthotics and Prostheses—Orthotic and prosthetic (O/P) devices in one instance may result in chafing, infection or skin abrasion where elements of the device rub against a skin surface. In other instances, the O/P device may result in impaired activities in underlying tissues, e.g. reduced blood flow, that may lead to unfavorable biological events, e.g. ulcerations. In yet other instances, the O/P devices may benefit from improved healing times of underlying tissues and/or reduction of pain thereby accelerating use of the device. The present invention may be utilized to provide therapeutic benefits for these conditions associated with the use of O/P devices. In one embodiment, these therapeutic systems are integrated within the O/P and in certain instances, may derive power from the operation of the O/P. In various embodiments, sensors may take the form of optical, electrical or electromagnetic sensors in order to locate and track body regions having inflammation, deep tissue reactions, etc. Likewise, therapeutic delivery may consist of photonic energies intended for primarily surface (skin or just beneath the skin) effects or one designed to penetrate deeper into the underlying tissues. These energies may be directed to act on the underlying bioprocess or indirectly act by use of one or more photosensitizing agents.
Adipose tissue remodeling—In certain circumstances, adipose tissue can be regulated through control of the degree of vascularization of these tissues. In one embodiment of the invention, a photoactivatable inhibitor of vascularization is supplied to a body zone. Upon determination by one or more sensors, e.g. tissue compositional sensors such as electrical impedance or ultra wideband radar, that a significant region of adipose tissue has been identified, a therapeutic light source might illuminate said region to result in the reduction of local blood supply and thereby reduce the amount of adipose tissue in this region. One such form of the invention might be as a wand moved over body zones enabling targeted applications to body regions to sculpt the underlying adipose tissues.
Muscle/tissue healing—Upon over exertion or strenuous work, one or more muscle groups and/or related structures such as joints, tendons might benefit from enhance healing provided by the device of the present invention. In such applications, the form of the device might be as a patch enabling placement upon a body zone that through sensors sensitivity to underlying changes associated with muscle/tissue over exertion, e.g. swelling, thereby enabling the targeted application of one or more photonic therapies. In one embodiment, the photonic therapy is a light energy suitable for deep penetration of body tissues and activation of the tissues to promote healing activities. In other forms of the invention, the device of the invention may be as a pad or mat whereupon the user places or lies upon in order to accomplish the desired therapeutic activity.

Additional forms and applications for the devices of the present invention are conceivable and the scope of the invention is not constrained to those examples presented above.

What is claimed is:

1) A method for application of one or more therapies to at least one body region where:
a) a plurality of body regions within a body zone are measured by one or more sensor elements;
b) the body region sensor data is analyzed by one or more comparators; and
c) at least one therapy may be applied to a body region based upon comparator analysis of body region sensor data.

2) The method of claim 1 where said sensor elements are photonic in nature.

3) The method of claim 1 where said therapy is photonic in nature.

4) The method of claim 1 where application of therapeutic photonic energies to individual body regions within the body zone occurs in an effectively simultaneous fashion.

5) The method of claim 1 where the applied photonic energies to one or more body regions may be the same or different from the applied photonic energies applied to one or more other body regions.

6) The method of claim 3 where the applied photonic energies may be of one or more light emission wavelengths, emission intensities and/or delivery patterns.

7) The method of claim 3 with additional, non-photonic therapies to one or more body regions in conjunction with application of one or more photonic therapies in response to analysis of body region sensor data.

8) The method of claim 1 where the sensing of a body zone is repeated over a period of time.

9) The method of claim 1 where said therapy to a body region is adjusted based upon one or more repeated sensor measurements.

10) The method of claim 1 where the sensor data of from one or more sensor measurements are mathematically evaluated thereby enabling trends of therapy efficacy in one or more body regions to be derived.

11) The method of claim 1 where the sensor data of from one or more sensor measurements are mathematically evaluated thereby enabling numbers of future repetitions and/or alterations of photonic therapy to individual body regions to be derived.

12) The method of claim 1 where the structure for the display of sensor data utilizes a structure physically separated from the structure utilized for sensor measurements and/or therapy delivery.

13) The method of claim 10 wherein communication between the display structure and the measurement and/or photonic therapy structures is accomplished by wireless means.

14) A device for the application of one or more therapies to individual body regions of a body zone consisting of:
a) a structure having one or more measurement sensors enabling the measurement of two or more body regions within said body zone for one or more parameters related to the status of said body regions;
b) a comparator capable of receiving and analyzing said measured data; and
c) a therapy delivery structure,
where said comparator evaluates body region parameter data from at least one measurement sensor and enables a delivery of at least one therapy to at least one body region using the therapy delivery structure upon said determination of need for said therapy.

15) The device of claim 14 where said sensors employ at least one wavelength of photonic energy for measurement of a body region.

16) The device of claim 14 where at least one wavelength of photonic energy as part of the applied therapy and said therapy delivery structure contains at least one photonic energy delivery source.

17) The device of claim 14 wherein the sensors, comparator and therapy delivery structure are contained within a single structure.

18) The multifunctional structure of claim 17 being effectively planar and flexible in nature and having a first surface with measurement elements and photonics sources oriented towards the body region.

19) The multifunctional structure of claim 17 also in wireless communication with one or more additional comparators.

20) The multifunctional structure of claim 17 wherein the measurement elements and photonic energy delivery sources form a geometrical arrangement having at least one repeated pattern of measurement elements and photonic energy delivery sources.

21) The multifunction structure of claim 17 having a first surface with measurement elements and photonics sources oriented towards the body region.

22) The multifunctional structure of claim 17 also having comparator and display functionalities.

23) The device of claim 14 where non-photonic therapies are supplied to one or more body regions in addition to photonic therapies.

24) A method for application of one or more therapies to a body zone where:
a) a plurality of body regions within a body zone are measured by one or more sensor elements;
b) the body region sensor data is analyzed by one or more comparators; and
c) one or more therapy(s) are applied to individual body regions based upon analysis of body region sensor data.

25) The method of claim 20 where application of therapies to individual body regions within the body zone includes the use of drugs or agents.
